(19) United States
(12) Patent Application Publication
(10) Pub. No.: US 2020/0158719 A1
(43) Pub. Date: May 21, 2020

(54) METHOD OF SPECIFYING CELL, METHOD OF PRODUCING CELL POPULATION AND CELL SPECIFYING SYSTEM

(71) Applicant: OLYMPUS CORPORATION, Tokyo (JP)

(72) Inventor: Ryutaro AKIYOSHI, Hachioji-shi (JP)

(73) Assignee: OLYMPUS CORPORATION, Tokyo (JP)

(21) Appl. No.: 16/773,126

(22) Filed: Jan. 27, 2020

Related U.S. Application Data
(63) Continuation of application No. PCT/JP2017/027514, filed on Jul. 28, 2017.

(51) Int. Cl. G01N 33/50; C12N 15/85; G01N 21/64

(52) U.S. Cl. CPC G01N 33/5094; G01N 2021/6439; G01N 21/6428; C12N 15/85

(57) ABSTRACT

A cell specifying method includes, for a first cell population including cells into which a nucleic acid encoding plural kinds of reprogramming factors necessary for reprogramming of somatic cells, and a nucleic acid encoding a luminescent reporter protein configured to be co-expressed with a kind of the plural kinds of reprogramming factors are introduced, acquiring a first luminescence image concerning luminescence caused by expression of the luminescent reporter protein, dividing the first cell population into second cell populations, for the second cell populations, acquiring a second luminescence image concerning luminescence caused by expression of the luminescent reporter protein, selecting objective cells from the first cell population based on the first luminescence image, and specifying the objective cells in the second cell populations based on the first luminescence image and the second luminescence image.

### CROSS-REFERENCE TO RELATED APPLICATIONS

This application is a Continuation Application of PCT Application No. PCT/JP2017/027514, filed Jul. 28, 2017, the entire contents of which are incorporated herein by reference.

### INCORPORATION BY REFERENCE OF SEQUENCE LISTING

The Sequence Listing in an ASCII text file, named as 38158Z_Sequence_Listing.txt of 5 KB, created on Jan. 24, 2020 and submitted to the United States Patent and Trademark Office via EFS-Web, is incorporated herein by reference.

### FIELD

Embodiments relate to a method of specifying cell, a method of producing cell population, and a cell specifying system.

### BACKGROUND

Induced pluripotent stem (iPS) cells are prepared by culturing somatic cells in which reprogramming factors (for example, Oct4, Klf4, Sox2, c-myc, Lin28 and L-myc) are introduced. Somatic cells into which reprogramming factors have been introduced are reprogrammed through a culture period. It is known that the efficiency of preparing iPS cells is low, and only part of cells into which reprogramming factors have been introduced become iPS cells.

In Eirini P. Papapetrou et al., Proc. Natl. Acad. Sci. USA. 2009 August; 106(31):12759-64. “Stoichiometric and temporal requirements of Oct4, Sox2, Klf4, and c-Myc expression for efficient human iPS induction and differentiation.”, reprogramming factors labeled with fluorescent proteins are expressed in cells, and the expression amounts of the reprogramming factors are analyzed. Papapetrou et al. reports that the reprogramming efficiency is improved when a vector harboring a reprogramming factor Oct4 is introduced into cells in an amount that is larger than the amounts of vectors harboring other reprogramming factors Sox2, Klf4, c-Myc, to express Oct4 in an amount larger than those of other reprogramming factors.

One cause of the low efficiency of preparation of iPS cells is ascribable to that iPS cells become easier to lose the pluripotency when the iPS cells are cultured for a long period.

Jpn. Pat. Appln. KOKAI Publication No. 2014-176364 describes a method for analyzing the state of a stem cell by using luminescence. The document describes a method for analyzing the differentiation state in the differentiation inducing stage from iPS cells to various organs by a promoter assay targeting a differentiation marker gene or an undifferentiation marker gene of cell. According to the method, it is possible to evaluate the uniformity of the differentiation state of individual iPS cells.

Since the efficiency of preparing iPS cells is low, it would be necessary to repeat subculture of iPS cells so as to establish a strain of iPS cells.

International Publication No. 2009/032194 describes a method for establishing iPS cells with high efficiency using a Wnt conditioned medium. The document indicates that iPS cells can be selected from a cell population in which reprogramming factors have been introduced, by utilizing flow cytometry, affinity separation or the like. According to the method, it is conceived that only iPS cells can be subcultured by flow cytometry, affinity separation or the like. However, separation of cells by flow cytometry, affinity separation or the like takes costs and labors.

For establishment of a strain of iPS cells, it is recommended to subculture cells situated in the center of a colony with excellent morphology among the colonies made up of cells into which reprogramming factors have been introduced. However, it is estimated that not all the cells in the center of the colony are necessarily iPS cells. Therefore, when the cells situated in the center of the colony are subcultured, iPS cells and cells other than iPS cells are undifferentially subcultured. When iPS cells and cells other than iPS cells are undifferentially subcultured, it is difficult to specify which cells are iPS cells after subculture without taking costs and labors.

### SUMMARY

According to an aspect, a cell specifying method comprises, for at least part of a first cell population including cells into which a nucleic acid encoding plural kinds of reprogramming factors necessary for reprogramming of somatic cells, and a nucleic acid encoding a luminescent reporter protein configured to be co-expressed with at least one kind of the plural kinds of reprogramming factors are introduced, acquiring a first luminescence image concerning luminescence caused by expression of the luminescent reporter protein. The specifying method comprises dividing the first cell population into a plurality of second cell populations. The specifying method comprises, for at least one of the second cell populations, acquiring a second luminescence image concerning luminescence caused by expression of the luminescent reporter protein. The specifying method comprises selecting objective cells from the first cell population based on the first luminescence image. The specifying method comprises specifying the objective cells in the plurality of second cell populations based on the first luminescence image and the second luminescence image.

Advantages of the embodiments will be set forth in the description which follows, and in part will be obvious from the description, or may be learned. The advantages may be realized and obtained by means of the instrumentalities and combinations particularly pointed out hereinafter.

### BRIEF DESCRIPTION OF THE DRAWINGS

The patent or application file contains at least one drawing executed in color. Copies of this paper or patent application publication with color drawing(s) will be provided by the Office upon request and payment of the necessary fee.

The accompanying drawings, which are incorporated in and constitute a part of the specification, illustrate embodiments, and together with the general description given above and the detailed description of the embodiments given below, serve to explain the principles.

FIG. 1 is a chart showing the flow of main treatments of a cell specifying method according to one embodiment.

FIG. 2 is a schematic diagram schematically showing S1 to S6 shown in FIG. 1.

FIG. 3A is a schematic diagram showing one example of a first luminescence image (color A).

FIG. 3B is a schematic diagram showing one example of a first luminescence image (color B).

FIG. 4A is a schematic diagram showing one example of a second luminescence image (color A).

FIG. 4B is a schematic diagram showing one example of a second luminescence image (color B).

FIG. 5A is a schematic diagram showing an example of difference in first luminescence intensity, and an example of difference in second luminescence intensity.

FIG. 5B is a schematic diagram showing an example of a first luminescence intensity ratio, and an example of a second luminescence intensity ratio.

FIG. 6A is a schematic diagram showing one example of a first luminescence image in which a first region of interest and a third region of interest are selected.

FIG. 6B is a schematic diagram showing one example of a second luminescence image in which a second region of interest and a fourth region of interest are selected.

FIG. 7 is a schematic diagram showing one example of a cell specifying system according to one embodiment.

FIG. 8 is a schematic diagram showing a configuration of vector A used in Example.

FIG. 9 is a schematic diagram showing a configuration of vector B used in Example.

FIG. 10A is a phase contrast image showing a first cell population.

FIG. 10B is a first luminescence image (green) showing luminescence originating in MA-Luci2 luciferase.

FIG. 10C is a first luminescence image (red) showing luminescence originating in SfRE1 luciferase.

FIG. 11 is a phase contrast image showing a second cell population.

FIG. 12A is a first luminescence image (green) shown in FIG. 10B in which a first region of interest (green) is selected.

FIG. 12B is a first luminescence image (red) shown in FIG. 10C in which a first region of interest (red) is selected.

FIG. 13 is a graph showing brightness (luminescence intensity) acquired for each of the first regions of interest (red) (610ALP) and (green) (BP495-540).

FIG. 14 is a graph showing first luminescence intensity ratio (Ratio (BP495-540 brightness/610ALP brightness)) determined from brightness (luminescence intensity) acquired for each of the first regions of interest (red) and (green).

FIG. 15A is a second luminescence image (green) showing luminescence originating in MA-Luci2 luciferase in which the second region of interest (green) is selected.

FIG. 15B is a second luminescence image (red) showing luminescence originating in SfRE1 luciferase in which the second region of interest (red) is selected.

FIG. 16 is a graph showing brightness (luminescence intensity) acquired for each of the second regions of interest (red) (610ALP) and (green) (BP495-540).

FIG. 17 is a graph showing second luminescence intensity ratio (Ratio (BP495-540 brightness/610ALP brightness)) determined from brightness (luminescence intensity) acquired for each of the second regions of interest (red) and (green).

FIG. 18A is a bright-field image showing objective cells at the time of starting the culture.

FIG. 18B is a bright-field image showing objective cells 5 hours after starting of the culture.

FIG. 18C is a bright-field image showing objective cells 10 hours after starting of the culture.

FIG. 18D is a bright-field image showing objective cells 16 hours after starting of the culture.

FIG. 18E is a bright-field image showing objective cells 20 hours after starting of the culture.

FIG. 18F is a bright-field image showing objective cells 22 hours after starting of the culture.

### DETAILED DESCRIPTION

Hereinafter, the embodiment will be described in detail, however, it is to be noted that the description aims at describing the present invention, but does not intend to limit the embodiment.

### 1. Description of Cell Specifying Method

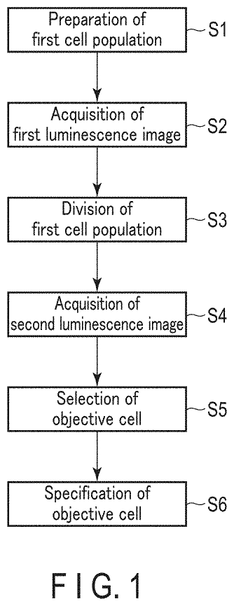

FIG. 1 shows the flow of main treatments of a cell specifying method according to one embodiment.

As shown in FIG. 1, a cell specifying method according to one embodiment includes:

preparing a first cell population including cells into which a nucleic acid encoding plural kinds of reprogramming factors necessary for reprogramming of somatic cells, and a nucleic acid encoding a luminescent reporter protein configured to be co-expressed with at least one kind of the plural kinds of reprogramming factors are introduced (S1);

acquiring a first luminescence image concerning luminescence caused by expression of the luminescent reporter protein for at least part of the first cell population (S2);

dividing the first cell population into a plurality of second cell populations (S3);

acquiring a second luminescence image concerning luminescence caused by expression of the luminescent reporter protein for at least one of the second cell populations (S4);

selecting objective cells from the first cell population based on the first luminescence image (S5); and

specifying the objective cells in the plurality of second cell populations based on the first luminescence image and the second luminescence image (S6).

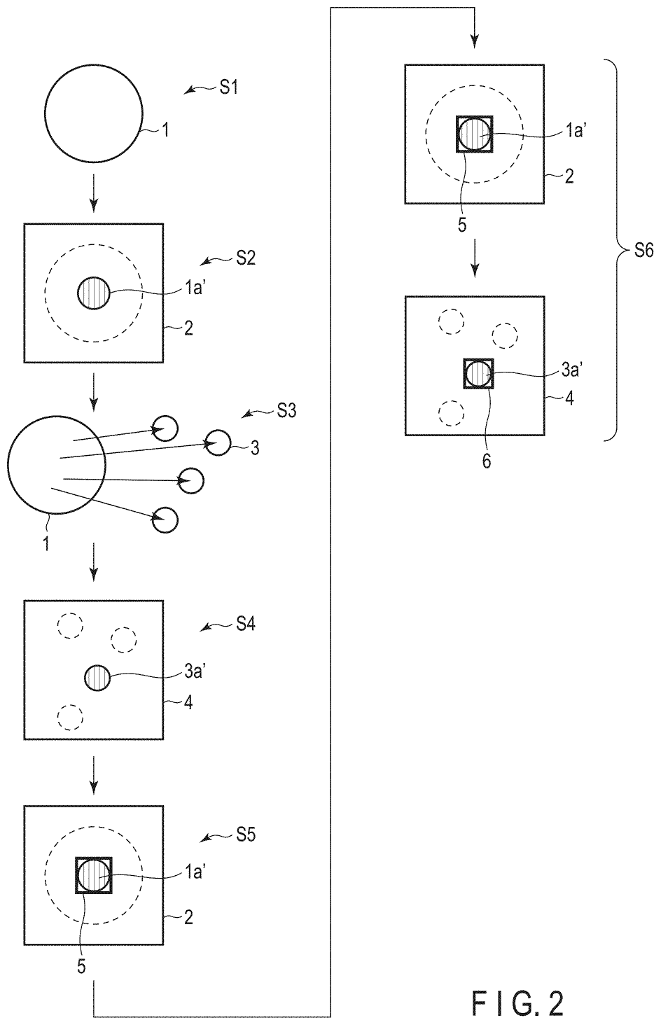

FIG. 2 is a schematic diagram schematically showing S1 to S6 shown in FIG. 1. Hereinafter, S1 to S6 are described by referring to FIG. 2.

<1-1. Preparation of First Cell Population>

As shown in S1 of FIG. 2, a first cell population 1 is prepared. The first cell population 1 includes cells into which a nucleic acid encoding plural kinds of reprogramming factors necessary for reprogramming of somatic cells, and a nucleic acid encoding a luminescent reporter protein configured to be co-expressed with at least one kind of plural kinds of reprogramming factors are introduced.

The first cell population 1 is obtained, for example, by culturing cells into which “a nucleic acid encoding plural kinds of reprogramming factors necessary for reprogramming of somatic cells”, and “a nucleic acid encoding a luminescent reporter protein configured to be co-expressed with at least one kind of plural kinds of reprogramming factors” are introduced. In the following description, the former nucleic acid is also referred to as “nucleic acid encoding reprogramming factors”, and the latter nucleic acid is also referred to as “nucleic acid encoding a luminescent reporter protein”. The nucleic acid is, for example, DNA. The nucleic acid may have the same meaning as a gene. The first cell population may be also expressed as a colony. The first cell population 1 may be one cell population among a plurality of cell populations obtained by culturing cells into which “nucleic acid encoding reprogramming factors”, and “nucleic acid encoding a luminescent reporter protein” are introduced.

Culture can be performed by a known method. Culture may be performed on the microscope except for the time for operation such as replacement of a culture medium. Culture may be continued, for example, until the minor axis of the first cell population 1 exceeds 1 mm. It is considered that somatic cells can reprogrammed with high efficiency when the culture is continued until the minor axis of the first cell population 1 exceeds 1 mm. “Reprogramming” means the phenomenon that a differentiated cell turns into a pluripotent stem cell, or initialization of a differentiated cell. The first cell population 1 has, for example, a major axis of 2 mm or less and a minor axis of 1 mm or more. The major axis of the first cell population 1 means the length where the distance of one straight line passing the center of gravity of the first cell population 1 and a point on the outer circumference of the first cell population 1 is maximum. The minor axis of the first cell population 1 means the length where the distance of the straight line is minimum.

In the description, the center of gravity of a cell population is defined as a point set in the following coordinates in a bright-field image acquired for the cell population.

$$( \frac{{\sum }_{i = 1}^{n} ( x i \cdot B i )}{{\sum }_{i = 1}^{n} B i} , \frac{{\sum }_{i = 1}^{n} ( yi \cdot B i )}{{\sum }_{i = 1}^{n} B i} )$$

In the coordinates, n represents the number of pixels constituting the bright-field image, xi represents the x coordinate in the i-th pixel, yi represents the y coordinate in the i-th pixel, and Bi represents a binarized value of brightness in the i-th pixel. Each of n and i is an integer of 1 or more. n and i satisfy n≥i. The center of gravity of the first cell population 1 can be set by using cellSens (OLYMPUS Corporation).

Culture may be continued, for example, up to 20 to 30 days after introduction of “nucleic acid encoding reprogramming factors” and “nucleic acid encoding a luminescent reporter protein” into somatic cells. Culture may be continued, for example, up to 20 to 25 days after introduction of “nucleic acid encoding reprogramming factors” and “nucleic acid encoding a luminescent reporter protein” into somatic cells. Culture is performed, for example, using a mononuclear cell proliferation culture medium in a plate coated with laminin. The mononuclear cell proliferation culture medium is, for example, a StemFit without C medium (AJINOMOTO CO., INC.) to which cytokines are added. As the culture medium, an AK02 medium (AJINOMOTO CO., INC.) to which IL-3, IL-6, SCF, TPO, Flt-3L, and CSF are added may be used.

The cells into which “nucleic acid encoding reprogramming factors” and “nucleic acid encoding a luminescent reporter protein” are introduced are, for example, somatic cells into which “nucleic acid encoding reprogramming factors” and “nucleic acid encoding a luminescent reporter protein” are introduced, or cultured cells of somatic cells into which “nucleic acid encoding reprogramming factors” and “nucleic acid encoding a luminescent reporter protein” are introduced. When somatic cells into which “nucleic acid encoding reprogramming factors” and “nucleic acid encoding a luminescent reporter protein” are introduced are cultured, reprogramming of somatic cells is induced by expression of “nucleic acid encoding reprogramming factors”. Therefore, the somatic cells can change into iPS cells.

The somatic cells are cells other than reproductive cells. The somatic cells are, for example, differentiated cells. Non-limiting examples of the somatic cells that can be used include human peripheral blood mononuclear cells (hereinafter, referred to as PBMC).

“Nucleic acid encoding reprogramming factors” and “nucleic acid encoding a luminescent reporter protein” may be introduced into somatic cells by a known gene introduction method. For example, “nucleic acid encoding reprogramming factors” and “nucleic acid encoding a luminescent reporter protein” may be incorporated into a vector, and the vector may be introduced into somatic cells by electroporation. For electroporation, Amaxa (Lonza) can be used.

“Nucleic acid encoding a luminescent reporter protein” is configured so that the luminescent reporter protein is co-expressed with at least one kind of plural kinds of reprogramming factors. Specifically, for example, “nucleic acid encoding a luminescent reporter protein” is configured so that “nucleic acid encoding a luminescent reporter protein” and “nucleic acid encoding reprogramming factors” are transcribed at the same timing. More specifically, it is preferred to couple each of “nucleic acid encoding a luminescent reporter protein” and “nucleic acid encoding reprogramming factors” to the same kind of promoter. “Nucleic acid encoding a luminescent reporter protein” and “nucleic acid encoding reprogramming factors” may be incorporated individually into separate vectors or incorporated into the same vector. It is preferred that the same kind of vector is used as the vector.

It is preferred that “nucleic acid encoding a luminescent reporter protein” and “nucleic acid encoding reprogramming factors” are incorporated into the same vector. It is preferred that “nucleic acid encoding a luminescent reporter protein” and “nucleic acid encoding reprogramming factors” are transcribed by the same promoter. When “nucleic acid encoding a luminescent reporter protein” and “nucleic acid encoding reprogramming factors” are transcribed by the same promoter, it is more preferred that “nucleic acid encoding a luminescent reporter protein” is configured to be polycistronically coupled with “nucleic acid encoding reprogramming factors” via a 2A sequence, IRES sequence or the like of a foot-and-mouth disease virus. It is preferred that “nucleic acid encoding a luminescent reporter protein” is configured to be polycistronically coupled with “nucleic acid encoding reprogramming factors” via a 2A sequence. “Nucleic acid encoding a luminescent reporter protein” may be configured to be coupled with “nucleic acid encoding reprogramming factors” via a linker sequence. As the linker sequence, a known sequence can be used. The linker sequence is composed, for example, of a plurality of nucleic acids encoding glycine and a plurality of nucleic acids encoding serine. The position of “nucleic acid encoding a luminescent reporter protein” can be any position at which the reprogramming factors and the luminescent reporter protein can be co-expressed, and may be upstream or downstream of “nucleic acid encoding reprogramming factors”.

“Nucleic acid encoding a luminescent reporter protein” may be configured so that the luminescent reporter protein is co-expressed with one kind of plural kinds of reprogramming factors, or may be configured so that the luminescent reporter protein is co-expressed with plural kinds of reprogramming factors. When the luminescent reporter protein is co-expressed with plural kinds of reprogramming factors, “nucleic acid encoding a luminescent reporter protein” and “nucleic acid encoding reprogramming factors” may be connected respectively via the aforementioned 2A sequence, IRES sequence, or linker sequence. Each of “nucleic acids encoding reprogramming factors” may be coupled via the aforementioned 2A sequence, IRES sequence, or linker sequence.

Since the luminescent reporter protein is co-expressed with the reprogramming factors, the timing of expression, the expression amount and the like of the gene encoding the luminescent reporter protein can be regarded as corresponding to those of the reprogramming factors.

“Plural kinds of nucleic acids encoding reprogramming factors” may be individually incorporated into separate vectors. Two or more nucleic acids among “plural kinds of nucleic acids encoding reprogramming factors” may be incorporated into one vector. When “plural kinds of nucleic acids encoding reprogramming factors” are incorporated into one vector, it is preferred that “plural kinds of nucleic acids encoding reprogramming factors” are polycistronically coupled via a 2A sequence, IRES sequence or the like of a foot-and-mouth disease virus. “Plural kinds of nucleic acids encoding reprogramming factors” may be coupled via a linker sequence.

As “plural kinds of reprogramming factors necessary for reprogramming of somatic cells”, a combination of reprogramming factors known to induce reprogramming of somatic cells can be used. As “plural kinds of reprogramming factors necessary for reprogramming of somatic cells”, for example, an appropriate combination can be selected from Oct3/4, Klf4, Sox2, c-myc, Lin28, and L-myc, and for example, a combination of Oct3/4, Klf4, Sox2, Lin28 and L-myc can be used. The number of kinds of reprogramming factors necessary for reprogramming of somatic cells is for example, three to six.

“Luminescent reporter protein” is, for example, a fluorescent protein or a chemiluminescent reporter protein.

A fluorescent protein is a protein that emits fluorescence by irradiation with an excitation light. When a fluorescent protein is used as the luminescent reporter protein, electrons of the fluorescent protein generated in cells by expression of the nucleic acid encoding the fluorescent protein come into an excited state by the excitation light applied to the cells, and emit fluorescence to come into a ground state. Therefore, it is possible to estimate the expression state or the like of the reprogramming factor co-expressed with the nucleic acid encoding the fluorescent protein based on the fluorescence. The fluorescent protein is, for example, green fluorescent protein (GFP), red fluorescent protein (RFP), yellow fluorescent protein (YFP), cyan fluorescent protein (CFP), Venus, mOrange, or mCherry.

A chemiluminescent reporter protein is a protein functioning to emit light without irradiation with excitation light. A chemiluminescent reporter protein causes luminescence by chemical reaction with a substrate. A chemiluminescent reporter protein is preferably a bioluminescent reporter protein, for example, luciferase.

When luciferase is used as the luminescent reporter protein, luciferase protein generated in cells by expression of luciferase gene reacts with luciferin provided for the cells, resulting in luminescence of luciferin. Therefore, it is possible to estimate the expression state or the like of the reprogramming factor co-expressed with luciferase according to the luminescence. Luminescence caused by expression of a chemiluminescent reporter protein can be observed without use of excitation light. Since observation without using excitation light is possible, it is possible to quantify the intensity of expression of reprogramming factors by a method that is minimally invasive to cells. A chemiluminescent reporter protein is difficult to cause cytotoxicity. Further, luminescence caused by expression of a chemiluminescent reporter protein is free of fading and excellent in quantifiability. A chemiluminescent reporter protein immediately matures as soon as expression is induced. Therefore, a chemiluminescent reporter protein is suited for reporting the expression state and the like of reprogramming factors. As the luciferin, any system including firefly luciferin, bacterial luciferin, dinoflagellate luciferin, vargulin, coelenterazine, and the like may be used.

As the luciferase, for example, various luciferases such as beetle luciferases including P. pyralis, click beetle, MA-Luci2, and SfRE1, marine luciferases including sea pansy luciferase, cypridina luciferase, aequorin, Copepoda luciferase, and luminescent shrimp luciferase, bacterial luciferases, and dinoflagellate luciferases can be used. Specifically, for example, as the luciferase, Eluc luciferase that provides green luminescence, CBR luciferase that provides red luminescence, and sea pansy luciferase that provides blue luminescence can be used. In particular, luciferase derived from firefly or luciferase derived from beetle such as click beetle are desired because such luciferase is ATP-demanding, and is not luminescent in a dead cell lacking the biological reaction, so that it is possible to selectively image living cells excluding the cells having devitalized due to apoptosis or the like during the culture period. As the reporter gene encoding the luminescent reporter protein, a commercially available gene can be used.

The luciferase used as the luminescent reporter protein may be modified luciferase that is modified to have high luminescence intensity. As the modified luciferase, for example, modified mutant A that is modified luciferase derived from Stenocladius flavipennis (Jpn. Pat. Appln. KOKAI Publication No. 2013-81459), luciferase derived from Pyrocoelia matsumurai (Jpn. Pat. Appln. KOKAI Publication No. 2014-18191) and the like can be used.

When there are two or more kinds (for example, two to six kinds) of reprogramming factors to be co-expressed with a luminescent reporter protein, each of the luminescent reporter proteins to be co-expressed with two or more kinds of reprogramming factors has such luminescence characteristics that allow detection of the protein distinguishably from any other luminescent reporter proteins. That is, two or more kinds of luminescent reporter proteins have such luminescence characteristics that allow detection of these proteins distinguishably from each other. Here, luminescence characteristics are, for example, luminescence wavelength. Thus, since two or more kinds of luminescent reporter proteins have such luminescence characteristics that allow detection of these proteins distinguishably from each other, information of luminescence caused by expression of each of the luminescent reporter proteins is distinguished from information of luminescence caused by expression of any other luminescent reporter proteins in the luminescence image acquired in the later step. Also, the luminescence intensity of luminescence caused by expression of each of the luminescent reporter proteins can be quantified individually (namely for each kind of luminescent reporter protein) based on the luminescence image.

As the two or more kinds of luminescent reporter proteins having such luminescence characteristics that allow detection of these proteins distinguishably from each other, for example, two or more kinds of proteins selected from the aforementioned chemiluminescent reporter proteins and fluorescent proteins can be used.

“Nucleic acid encoding reprogramming factors” can be introduced into somatic cells, for example, by using a vector set including a first vector containing a nucleic acid encoding Oct3/4, a second vector containing a nucleic acid encoding Sox2 and a nucleic acid encoding Klf4, and a third vector containing a nucleic acid encoding L-myc and a nucleic acid encoding Lin28. Here, at least one of the first vector, the second vector, and the third vector contains “nucleic acid encoding a luminescent reporter protein” at a position capable of being co-expressed with “nucleic acid encoding reprogramming factors” contained in the vector.

In addition to “nucleic acid encoding reprogramming factors” and “nucleic acid encoding a luminescent reporter protein”, “nucleic acid encoding an additional factor that enhances the reprogramming efficiency” may be introduced into somatic cells. As “additional factor that enhances the reprogramming efficiency”, a factor that is known to enhance the reprogramming efficiency, for example, mouse p53, EBNA1 into which dominant negative mutation is introduced can be used.

When “nucleic acid encoding reprogramming factors” is introduced using the vector set including the first to third vectors, a fourth vector including a nucleic acid encoding mouse p53 into which dominant negative mutation is introduced and fifth vector including a nucleic acid encoding EBNA1 into which dominant negative mutation is introduced are further incorporated into the vector set.

It is preferred that “nucleic acid encoding reprogramming factors” and “nucleic acid encoding a luminescent reporter protein” are not incorporated into a genome of the host, but introduced in such a form that the nucleic acids are expressed continuously. For example, it is preferred that “nucleic acid encoding reprogramming factors” and “nucleic acid encoding a luminescent reporter protein” are introduced in somatic cells in the form of an episomal vector. When these nucleic acids are introduced into somatic cells in the form of a episomal vector, these nucleic acids come off from the cells during culture, so that iPS cells not containing both “nucleic acid encoding reprogramming factors” and “nucleic acid encoding a luminescent reporter protein” can be obtained. In this case, as the episomal vector, a commercially available episomal vector may be used, or a modified vector prepared by incorporating “nucleic acid encoding a luminescent reporter protein” into a commercially available episomal vector may be used. Examples of the commercially available episomal vector containing reprogramming factors that can be used include pCXLE-hOCT3/4-shp53-F (Addgene), pCXLE-hSK (Addgene), and pCXLE-hUL (Addgene).

It is preferred that the aforementioned “additional factor that enhances the reprogramming efficiency” is also introduced into somatic cells in the form of an episomal vector. In this case, as the episomal vector, those commercially available, for example, pCE-mp53DD (Addgene), and pCXB-EBNA1 (Addgene) can be used.

<1-2. Acquisition of First Luminescence Image>

As shown in S2 of FIG. 2, for at least part of the first cell population 1, the first luminescence image 2 concerning luminescence caused by expression of a luminescent reporter protein is acquired. In S2 of FIG. 2, the dashed line indicates the outer circumference of the first cell population 1, and a high luminescence intensity cell 1a′ is one or more cell(s) showing high luminescence intensity.

The first luminescence image 2 is one or more luminescence image(s) obtained by imaging at least part of the first cell population 1. The first luminescence image 2 may be one luminescence image, or may be a plurality of luminescence images. When the first luminescence image 2 is a plurality of luminescence images, one or more luminescence image(s) selected from the plurality of luminescence images may be used as the first luminescence image 2 in the later-described selection of objective cells. Each of the plurality of luminescence images may be, for example, an image obtained by imaging a region different from any other luminescence images, or may be a plurality of luminescence images that are imaged over time.

Preferably, the luminescence image is acquired by imaging the entire first cell population 1. The luminescence image may be acquired by imaging part of the first cell population 1.

The acquiring the luminescence image preferably includes adjusting the focal position of imaging to the inside of the first cell population 1. By adjusting the focal position of imaging to the inside of the first cell population 1, it is possible to observe luminescence originating in a cell existing inside the first cell population 1.

It is preferred that the acquiring the luminescence image is performed in a light-shielded environment. More specifically, it is preferred that the acquiring the luminescence image is performed in a light-shielded environment for imaging luminescence by a method that is less influenced from outside. The luminescence image can be acquired by appropriately using a filter in a light-shielded environment.

When the luminescent reporter protein to be detected is one kind of chemiluminescent reporter protein, the first luminescence image 2 may be acquired in a light-shielded environment without using a filter.

In at least one of the case where there are plural kinds of luminescent reporter proteins to be detected, and the case where the luminescent reporter protein to be detected is a fluorescent protein, it is preferred that the luminescence image is acquired after spectral diffraction by appropriately using a filter in a light-shielded environment. The luminescence image can also be acquired as an image in which luminescence caused by expression of plural kinds of luminescent reporter proteins is overlapped without conducting spectral diffraction with a filter. The luminescence image can also be acquired as a color image using a color CCD camera or a color CMOS camera. The luminescence image may be acquired by macro-imaging distribution of luminescence amount in a wide region (region embracing 50 to 500 colonies) by in vivo imaging, followed by micro-scoping of only a site of interest. The luminescence image may be acquired using a cooled CCD camera.

Hereinafter, one example of an imaging method in the case where the luminescent reporter protein is a fluorescent protein is described. First, the first cell population 1 is irradiated with excitation light. The excitation light is applied by a light source, and an excitation light filter that separates excitation light from the light radiated from the light source. Then, cells in the first cell population 1 emit fluorescence. Then, the fluorescence is imaged with a camera via a filter that separates fluorescence.

The exposure time of the camera is, for example, 10 milliseconds to 1 second, and can be appropriately adjusted so that a sufficient fluorescent signal can be detected.

The luminescence image may be obtained by repeatedly conducting imaging over time. Imaging is conducted contiguously at any intervals. Imaging is generally conducted at intervals of 10 to 30 minutes. The time of one imaging is set at will. The interval at which the imaging is conducted is any time interval that is longer than the light exposure time required for generation of a luminescence image that can be analyzed by an image pickup device. In the above, the imaging method in the case where the luminescent reporter protein is a fluorescent protein has been described.

Hereinafter, one example of an imaging method in the case where the luminescent reporter protein is a chemiluminescent reporter protein is described. It is assumed that the chemiluminescent reporter protein is luciferase. First, the first cell population 1 is provided with luciferin. Specifically, for example, luciferin is added to a container containing the first cell population 1. Then, the luciferin transfers into cells and the luciferin reacts with luciferase in the cells, and thus the luciferin emits chemical luminescence. Then, the chemical luminescence is imaged with a camera via a filter that separates the chemical luminescence.

The camera exposure time is, for example, 3 to 5 minutes, and can be appropriately adjusted so that a sufficient luminescent signal can be detected.

The luminescence image may be obtained by repeatedly conducting imaging over time. Imaging is conducted contiguously at any intervals. Imaging is generally conducted at intervals of 10 to 30 minutes, for example, at intervals of 10 minutes. The time of one imaging is set at will. The interval at which the imaging is conducted is any time interval that is longer than the exposure time required for generation of a luminescence image that can be analyzed by an image pickup device. In the above, the imaging method in the case where the luminescent reporter protein is a chemiluminescent reporter protein has been described.

The luminescence image can be acquired by using a luminescence imaging device. The luminescence imaging device includes, for example, a filter that transmits mainly the light having a specific wavelength depending on the luminescence, an image pickup device that converts the light having transmitted the filter into an electric signal, and a processor that creates a luminescence image from the electric signal. By executing the imaging function at a desired timing while the culture function possessed by the luminescence imaging device is executed, it is possible to acquire luminescence images in the entire step of culture. As the luminescence imaging device, a later-described luminescence image generating apparatus or a luminescence imaging system can be used. The luminescence imaging device is, for example, a luminescence imaging system LV200 (OLYMPUS Corporation).

Preferably, a bright-field image is acquired in addition to the luminescence image. More preferably, a bright-field image is acquired almost the same timing with acquisition of the luminescence image. A bright-field image means an image that is acquired by using illumination light without based on luminescence, and is an image in which position, morphology and the like of cells or colonies can be observed. The bright-field image includes a phase contrast observation image and a differential interference contrast (DIC) observation image. The bright-field image may be acquired at almost the same timing as acquisition of a luminescence image, or may be acquired in any timing independently of acquisition of a luminescence image. In an automated system such as the luminescence imaging system (LV200), it is also possible to execute both the imaging function for a luminescence image and the imaging function for a bright-field image while switching these functions at a prespecified timing, under the sufficient light-shielded condition.

Preferably, the first luminescence image 2 is one image that is selected from a plurality of luminescence images, and each of the plurality of luminescence images is an image showing a plane at a position that is different from a position of any other luminescence images along a thickness direction of the first cell population 1. Cells on the surface of the first cell population 1, and cells inside the first cell population 1 sometimes show different luminescence.

Therefore, by acquiring the plurality of luminescence images, it is possible to select a later-described objective cells from cells on the surface of the first cell population 1 and cells inside the first cell population 1.

<1-3. Division of First Cell Population>

As shown in S3 of FIG. 2, the first cell population 1 is divided into a plurality of second cell populations 3.

The dividing the first cell population 1 into the plurality of second cell populations 3 is, for example, division of the first cell population 1 into a plurality of subpopulations. The dividing the first cell population 1 into the plurality of second cell populations 3 may be conducted, for example, by stirring a cell-containing liquid containing the first cell population 1 and a liquid, or by laser microdissection. The stirring the cell-containing liquid is, for example, pipetting the cell-containing liquid. The cell-containing liquid is pipetted to give a cell suspension. The cell suspension may be discharged to a plate. The liquid may be a StemFit+Y medium. The StemFit+Y medium is a medium prepared by adding Y-27632 to StemFit (AJINOMOTO CO., INC.).

When the first cell population 1 is divided into the plurality of second cell populations 3, at least one second cell population 3 contains a high luminescence intensity cell 1a′. The high luminescence intensity cell 1a′ is, for example, a later-described objective cell. When the first cell population 1 is divided into the plurality of second cell populations 3, grasping of the position of the objective cells in the second cell population 3 tends to be difficult.

The dividing the first cell population 1 into the plurality of second cell populations 3 is, preferably, seeding the later-described objective cells. Specifically, the seeding the objective cells is, for example, sucking up a part containing the objective cells in the first cell population 1 with a pipette, pipetting the sucked up part in a new liquid, and discharging the cell suspension of the sucked part and the liquid to a plate. Pipetting the sucked up part in the liquid results in division of the sucked part into the plurality of second cell populations 3. Therefore, discharging the cell suspension to a plate means discharging the plurality of second cell populations 3 to a plate. After seeding of the objective cells, the plurality of second cell populations 3 may be cultured. In other words, the dividing the first cell population 1 into the plurality of second cell populations 3 may be seeding the objective cells, and culturing the plurality of second cell populations 3.

Each of the second cell populations 3 contains, for example, several to several tens of cells. Preferably, each of the second cell populations 3 contains three to ten cells. When the number of cells contained in the second cell population 3 is small, the second cell population 3 is difficult to proliferate. If the number of cells contained in the second cell population 3 is large, there is high possibility that a lot of cells other than the later-described objective cell are contained, and thus it can be difficult to establish a strain of iPS cells when the objective cells are iPS cells.

The divided second cell populations 3 may be discharged to other plate than the plate containing the first cell population 1. As the plate, a plate coated with laminin as described above may be used.

<1-4. Acquisition of Second Luminescence Image>

As shown in S4 of FIG. 2, for at least one second cell population 3, the second luminescence image 4 concerning luminescence caused by expression of a luminescent reporter protein is acquired. In S4 of FIG. 2, the dashed line indicates the outer circumference of the second cell population 3. In the second luminescence image 4, a high luminescence intensity cell 3a′ is one or more cell(s) showing high luminescence intensity.

The second luminescence image 4 is one or more luminescence image (s) obtained by imaging at least one second cell population 3. The acquiring the second luminescence image 4 can be conducted in the same method as described in the section of “<1-2. Acquisition of first luminescence image>”.

It is preferred that the time from acquisition of the first luminescence image 2 to acquisition of the second luminescence image 4 is short. That is, it is preferred that the luminescence profile of the objective cells at the time of acquiring the first luminescence image 2 does not change at the point of time of acquiring the second luminescence image 4. Specifically, the time from acquisition of the first luminescence image 2 to acquisition of the second luminescence image 4 ranges from 5 to 60 minutes. Therefore, it is possible to specify the objective cells in the plurality of second cell populations 3 based on the first luminescence image 2 and the second luminescence image 4.

Acquisition of the second luminescence image 4 may be conducted for every second cell population 3, or may be conducted for part of the second cell populations among the plurality of second cell populations 3.

<1-5. Selection of Objective Cells>

As shown in S5 of FIG. 2, objective cells are selected from the first cell population 1 based on the first luminescence image 2. Here, the high luminescence intensity cell 1a′ was selected as the objective cell.

The objective cells are cells showing luminescence caused by expression of a luminescent reporter protein. The objective cells are, for example, iPS cells. The objective cells are selected, for example, based on the luminescence profile of each cell in the first luminescence image 2. The luminescence profile is, for example, a luminescence intensity, a luminescence intensity distribution or a combination of these. The luminescence intensity distribution is, for example, a distribution of a luminescence region in a cell. For example, as the objective cells, cells showing strong luminescence intensity may be selected, and for example, cells showing strong luminescence intensity in the nucleus may be selected. The objective cells may be selected based on the luminescence profile, or may be selected by visual observation. When the luminescence image is acquired over time, the objective cells may be selected based on the temporal luminescence intensity, the temporal luminescence intensity distribution, or the combination of these.

As shown in S5 of FIG. 2, preferably, the selecting objective cells includes selecting a first region of interest 5 concerning the objective cells on the first luminescence image 2. More preferably, the selecting objective cells includes selecting the first region of interest 5 concerning objective cells on the first luminescence image 2, and acquiring a first luminescence profile concerning the first region of interest 5. The selecting the first region of interest 5 concerning objective cells is, in other words, selection of the first region of interest 5 concerning a region containing the objective cell. Preferably, 90% or more of the first region of interest 5 is cells. Acquisition of the first luminescence profile may be conducted immediately after selection of the first region of interest 5, or may be conducted at the time of acquiring the second luminescence profile in the later-described “<1-6. Specification of objective cells>”.

It is preferred that the first region of interest 5 contains three to ten cells. It is preferred that the number of cells contained in the first region of interest 5 coincides with the number of cells contained in a later-described second region of interest 6. When these numbers are identical, the later-described evaluation of the first region of interest 5 and the later-described second region of interest 6 can be conducted with high reliability. Preferably, the first region of interest 5 does not contain a lot of cells other than the objective cell. When the first region of interest 5 contains a lot of cells other than the objective cell, it can become difficult to specify the objective cells. The first region of interest 5 is, for example, a region having the highest brightness. The shape of the first region of interest 5 is not particularly limited, but is, for example, a rectangle.

The first region of interest 5 is located inside a region including the center of gravity and the vicinity thereof of the first cell population 1. The vicinity of the first cell population 1 is, for example, a region within the range of 100 to 1,000 μm from the center of gravity of the first cell population 1.

The selecting objective cells from the first cell population 1 preferably includes imaging a bright-field image of the first cell population 1, and selecting objective cells from a region within 1 mm from the center of gravity of the first cell population 1 that is set based on the bright-field image. The selecting objective cells from the region within 1 mm from the center of gravity of the first cell population 1 may be conducted before selection of objective cells based on the first luminescence image 2. That is, objective cells may be selected from the region within 1 mm from the center of gravity of the first cell population 1 based on the first luminescence image 2. It is highly possible that a cell located near the center of gravity of the first cell population 1 is an iPS cell. The center of gravity of the first cell population 1 described herein is the center of gravity of the colony.

Two or more first regions of interest 5 may be selected.

Selection of objective cells may be conducted after division of the first cell population 1, or may be conducted before division of the first cell population 1.

Hereinafter, an example of selecting objective cells is described.

[First Example of Selecting Objective Cells]

Hereinafter, one example of selecting objective cells from the first cell population 1 based on the first luminescence image 2 is described. In this example, the luminescent reporter protein is one kind. Also, in this example, the selecting objective cells includes selecting the first region of interest 5 concerning objective cells on the first luminescence image 2, and acquiring a first luminescence profile concerning the first region of interest 5.

(i) Selection of First Region of Interest

First, the first region of interest 5 concerning objective cells is selected on the first luminescence image 2. The area of the first region of interest ranges, for example, from 0.01 to 1 mm².

(ii) Acquisition of First Luminescence Profile

Then, for the first region of interest 5, the first luminescence profile is acquired.

The first luminescence profile is, for example, a luminescence intensity, a luminescence intensity distribution, an evaluation value of luminescence intensity distribution or a combination of these. The luminescence intensity is a luminescence intensity detected in a wavelength range of luminescence of luminescent reporter protein. The luminescence intensity distribution is, for example, a distribution of a luminescence region in a cell. The evaluation value of luminescence intensity distribution is, for example, an evaluation value of distribution evaluating the luminescence intensity distribution. Preferably, the first luminescence profile is luminescence intensity. The first luminescence profile can be acquired, for example, by using existing image analysis software. The image analysis software is, for example, cellSens (OLYMPUS Corporation).

In the above, one example of selecting objective cells from the first cell population 1 based on the first luminescence image 2 has been described.

[Second Example of Selecting Objective Cells]

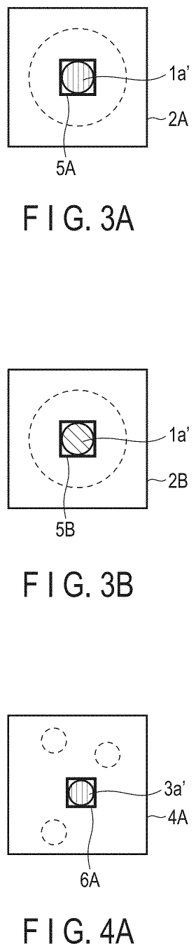

Hereinafter, with reference to FIG. 3A and FIG. 3B, another example of selecting objective cells from the first cell population 1 based on the first luminescence image is described. In this example, there are two kinds of luminescent reporter proteins, and the first luminescence image is separated with a filter to give two luminescence images respectively acquired for predetermined wavelength ranges. The two luminescence images are a first luminescence image (color A) and a first luminescence image (color B). The two kinds of luminescent reporter proteins are a first luminescent reporter protein and a second luminescent reporter protein.

FIG. 3A is a schematic diagram showing one example of a first luminescence image (color A) 2A. The first luminescence image (color A) 2A is the first luminescence image regarding color A. Color A is a color of luminescence caused by expression of the first luminescent reporter protein. In FIG. 3A, the high luminescence intensity cell 1a′ is one or more cell(s) showing high luminescence intensity, and the dashed line indicates the outer circumference of the first cell population 1.

FIG. 3B is a schematic diagram showing one example of a first luminescence image (color B) 2B. The first luminescence image (color B) 2B is the first luminescence image regarding color B. Color B is a color of luminescence caused by expression of the second luminescent reporter protein. In FIG. 3B, the high luminescence intensity cell 1a′ is one or more cell(s) showing high luminescence intensity, and the dashed line indicates the outer circumference of the first cell population 1.

Both of the first luminescence image (color A) 2A and the first luminescence image (color B) 2B are luminescence images having the same imaging range. Also, in this example, the selecting objective cells includes selecting the first region of interest 5 concerning objective cells on the first luminescence image 2, and acquiring a first luminescence profile concerning the first region of interest 5.

(i) Selection of First Region of Interest

First, the first region of interest 5 concerning objective cells is selected on the first luminescence image 2. Specifically, a first region of interest (color A) 5A is selected in the first luminescence image (color A) 2A, and a first region of interest (color B) 5B is selected in the first luminescence image (color B) 2B. The first region of interest (color A) 5A and the first region of interest (color B) 5B show luminescence having wavelength ranges that are different from each other. The first region of interest (color A) 5A and the first region of interest (color B) 5B have the same imaging range. At least one of the first region of interest (color A) 5A and the first region of interest (color B) 5B is a light-emitting region.

(ii) Acquisition of First Luminescence Profile

Then, for the first region of interest, the first luminescence profile is acquired. Preferably, the first luminescence image (color A) 2A and the first luminescence image (color B) 2B are subjected to an unmixing process to exclude the part where the wavelength ranges of luminescence of the two kinds of luminescent reporter proteins overlap with each other, and then the first luminescence profile is acquired.

Specifically, for example, the first luminescence profile is acquired for the first region of interest (color A) 5A and the first region of interest (color B) 5B. According to one example, the first luminescence profile is a luminescence intensity for each of the first region of interest (color A) 5A and the first region of interest (color B) 5B, an evaluation value of luminescence intensity distribution for each of the first region of interest (color A) 5A and the first region of interest (color B) 5B, or a combination of these. According to other example, the first luminescence profile is a later-described first luminescence intensity ratio, a total luminescence intensity that is the sum total of luminescence intensity for each of the first region of interest (color A) 5A and the first region of interest (color B) 5B, an evaluation value ratio obtained from evaluation values of luminescence intensity distribution for each of the first region of interest (color A) 5A and first region of interest (color B) 5B, or a combination of these. The first luminescence intensity ratio is a luminescence intensity ratio determined from each luminescence intensity caused by expression of each of the luminescent reporter proteins. Preferably, the first luminescence profile is a luminescence intensity caused by expression of each of the luminescent reporter proteins.

In the above, another example of selecting objective cells from the first cell population based on the first luminescence image has been described.

<1-6. Specification of Objective Cells>

In S6 of FIG. 2, objective cells in the plurality of second cell populations 3 are specified based on the first luminescence image 2 and the second luminescence image 4. The first luminescence image 2 shown in S6 is the first luminescence image 2 shown in S5. The second luminescence image 4 shown in S6 is the second luminescence image 4 shown in S4.

The specifying objective cells in the plurality of second cell populations 3 based on the first luminescence image 2 and the second luminescence image 4 is, for example, specifying objective cells in a set of the plurality of second cell populations 3 in the second luminescence image 4, based on, for example, a luminescence profile of objective cells in the first luminescence image 2, and a luminescence profile of cells in the second cell population 3. The luminescence profile is, for example, “luminescence profile” described in the section of “<1-5. Selection of objective cells>”.

To be more specific, the specifying objective cells in the plurality of second cell populations 3 based on the first luminescence image 2 and the second luminescence image 4 includes,

selecting a second region of interest 6 concerning the second cell population 3 on the second luminescence image 4, and

specifying objective cells from the plurality of second cell populations 3 based on the first luminescence profile acquired for the first region of interest 5 and the second luminescence profile acquired for the second region of interest 6.

More specifically, it is preferred that the specifying objective cells in the plurality of second cell populations 3 based on the first luminescence image 2 and the second luminescence image 4 includes,

(i) selecting a second region of interest concerning the second cell population 3 on the second luminescence image 4,

(ii) acquiring the second luminescence profile for the second region of interest, and

(iii) specifying objective cells in the plurality of second cell populations based on the first luminescence profile and the second luminescence profile.

Hereinafter, an example of specifying objective cells is described.

[First Example of Specifying Objective Cells]

Hereinafter, one example of specifying objective cells in the plurality of second cell populations 3 based on the first luminescence image 2 and the second luminescence image 4 is described. In this example, the luminescent reporter protein is one kind. In this example, the selecting objective cells was conducted in the same manner as described in “[First example of selecting objective cells]”.

(i) Selection of Second Region of Interest

As shown in S6 of FIG. 2, first, the second region of interest 6 concerning the second cell population 3 is selected on the second luminescence image 4. That is, on the second luminescence image 4, the second region of interest 6 is selected for the second cell population 3. In S6 of FIG. 2, the second region of interest 6 concerning the high luminescence intensity cell 3a′ was selected. The second region of interest 6, for example, little contains cells other than the second cell population 3. Preferably, 90% or more of the second region of interest 6 is the second cell population 3. The selecting the second region of interest 6 concerning the second cell population 3 is, in other words, selection of the second region of interest 6 as a region containing the second cell population 3. One second region of interest 6 is a region containing one or more cell(s) in the second cell population 3. One second region of interest 6 may be a region containing the whole of the second cell population 3. There may be two or more second regions of interest. As the two or more second regions of interest 6, the second region of interest 6 concerning each of the two or more second cell populations 3 may be selected. Two or more second regions of interest 6 concerning one second cell population 3 may be selected. The second region of interest 6 may be determined by visual observation. The shape of the second region of interest 6 is not particularly limited, but is, for example, a rectangle.

Preferably, the second region of interest 6 contains three to ten cells. It is preferred that the number of cells contained in the second region of interest 6 coincides with the number of cells contained in the first region of interest 5. When these numbers are identical, the later-described evaluation of the first region of interest 5 and the second region of interest 6 can be conducted with high reliability.

(ii) Acquisition of Second Luminescence Profile

Then, for the second region of interest 6, the second luminescence profile is acquired. The second luminescence profile includes the same kind of luminescence profile as the first luminescence profile. The second luminescence profile can be acquired, for example, by using existing image analysis software.

(iii) Specification of Objective Cells

Then, objective cells in the plurality of second cell populations 3 are specified based on the first luminescence profile and the second luminescence profile. Specifically, when one first luminescence profile and one second luminescence profile coincide with each other, it is evaluated that the relevant first region of interest 5 and the relevant second region of interest 6 correspond with each other. Since the first region of interest 5 and the second region of interest 6 correspond with each other, cells in the first region of interest 5 and cells in the second region of interest 6 also correspond with each other. The first region of interest 5 contains objective cells, and cells in the second region of interest 6 are cells in the second cell population 3. Therefore, the objective cells and the cells in the second cell population 3 correspond with each other. Therefore, when the first region of interest 5 and the second region of interest 6 correspond with each other, it is determined that the objective cells and the cells in the second cell population 3 are the same with each other. By the method as described above, the objective cells in the plurality of second cell populations 3 are specified.

On the other hand, when one first luminescence profile and one second luminescence profile do not coincide with each other, it is evaluated that the relevant first region of interest 5 and the relevant second region of interest 6 do not correspond with each other. Since the first region of interest 5 and the second region of interest 6 do not correspond with each other, cells in the first region of interest 5 and cells in the second region of interest 6 also do not correspond with each other. The first region of interest 5 contains objective cells, and cells in the second region of interest 6 are cells in the second cell population 3. Therefore, the objective cells and the cells in the second cell population 3 do not correspond with each other. Therefore, when the first region of interest 5 and the second region of interest 6 do not correspond with each other, it can be determined that the objective cells and the cells in the second cell population 3 are not the same cells. According to the method as described above, it can be recognized that the objective cells do not exist in the second region of interest 6.

Specifically, whether the first luminescence profile and the second luminescence profile coincide with each other can be determined based on the difference between the first luminescence profile and the second luminescence profile. The difference between the first luminescence profile and the second luminescence profile is, for example, the absolute value of the value obtained by dividing the difference between the first luminescence profile and the second luminescence profile by the first luminescence profile, and multiplying the resultant value by 100. That is, the difference between the first luminescence profile and the second luminescence profile is indicated in percent.

The difference between the first luminescence profile and the second luminescence profile is, for example, difference in luminescence intensity, difference in evaluation value of distribution, or a combination of these.

In the case where the difference between the first luminescence profile and the second luminescence profile is the difference in luminescence intensity, it is determined that the first luminescence profile and the second luminescence profile coincide with each other when the difference between the first luminescence profile and the second luminescence profile is 10% or less. On the other hand, when the difference between the first luminescence profile and the second luminescence profile is larger than 10%, it is determined that the first luminescence profile and the second luminescence profile do not coincide with each other.

In the above, one example of specifying objective cells in the plurality of second cell populations 3 based on the first luminescence image and the second luminescence image has been described.

[Second Example of Specifying Objective Cells]

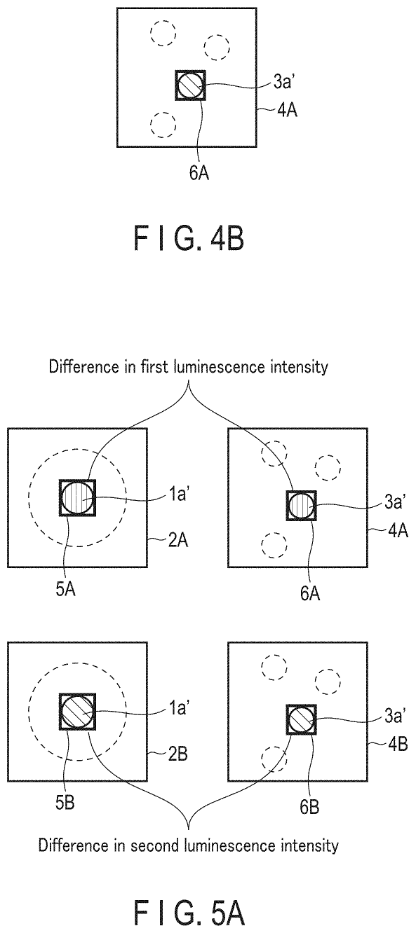

Hereinafter, referring to FIG. 4A and FIG. 4B, another example of specifying objective cells in the plurality of second cell populations 3 based on the first luminescence image 2 and the second luminescence image 4 is described. In this example, there are two kinds of luminescent reporter proteins, and the second luminescence image 4 is also two luminescence images respectively acquired for predetermined wavelength ranges. Two luminescence images are a second luminescence image (color A) and a second luminescence image (color B). The two kinds of luminescent reporter proteins are a first luminescent reporter protein and a second luminescent reporter protein.

FIG. 4A is a diagram showing one example of a second luminescence image (color A) 4A. The second luminescence image (color A) 4A is the second luminescence image 4 regarding color A. Color A is a color of luminescence caused by expression of the first luminescent reporter protein. In FIG. 4A, the high luminescence intensity cell 3a′ is one or more cell(s) showing high luminescence intensity, and the dashed line indicates the outer circumference of the second cell population 3.

FIG. 4B is a schematic diagram showing one example of a second luminescence image (color B) 4B. The second luminescence image (color B) 4B is the second luminescence image 4 regarding color B. Color B is a color of luminescence caused by expression of the second luminescent reporter protein. In FIG. 4A, the high luminescence intensity cell 3a′ is one or more cell(s) showing high luminescence intensity, and the dashed line indicates the outer circumference of the second cell population 3.

Both of the second luminescence image (color A) 4A and the second luminescence image (color B) 4B are luminescence images having the same imaging range. In this example, the selecting objective cells was conducted in the same manner as described in “[Second example of selecting objective cells]”.

(i) Selection of Second Region of Interest

First, the second region of interest 6 concerning the second cell population 3 is selected on the second luminescence image 4. Specifically, one or more second region(s) of interest (color A) 6A is selected in the second luminescence image (color A) 4A, and at least one second region of interest (color B) 6B is selected in the second luminescence image (color B) 4B. The second region of interest (color A) 6A and the second region of interest (color B) 6B show luminescence having wavelength ranges that are different from each other. The second region of interest (color A) 6A and the second region of interest (color B) 6B have the same imaging range. The second region of interest (color A) and the second region of interest (color B) may be “second region of interest 6” described in “(i) Selection of second region of interest” in “[First example of specifying objective cells]”.

(ii) Acquisition of Second Luminescence Profile

Then, for the second region of interest 6, the second luminescence profile is acquired. Specifically, for example, the second luminescence profile is acquired for the second region of interest (color A) 6A and the second region of interest (color B) 6B. The second luminescence profile includes the same kind of luminescence profile as the first luminescence profile. The second luminescence profile is, for example, a luminescence intensity ratio determined from each luminescence intensity caused by expression of each of the luminescent reporter proteins, namely, a second luminescence intensity ratio. Preferably, the second luminescence profile is a luminescence intensity caused by expression of each of the luminescent reporter proteins.

(iii) Specification of Objective Cells

Then, objective cells in the plurality of second cell populations 3 are specified based on the first luminescence profile and the second luminescence profile. Specifically, as described in “(iii) Specification of objective cell” in “[First example of specifying objective cells]”, when one first luminescence profile and one second luminescence profile coincide with each other, it is evaluated that the relevant first region of interest 5 and the relevant second region of interest 6 correspond with each other, and it is determined that the objective cells and the cells in the second cell population 3 are the same, and thus the objective cells in the plurality of second cell populations 3 is specified. On the other hand, when the first luminescence profile and the second luminescence profile do not coincide with each other, it is evaluated that the relevant first region of interest 5 and relevant second region of interest 6 do not correspond with each other, and it is determined that the objective cells are not the same cells as the cells in the second cell population 3, and thus it is confirmed that the objective cells do not exist in the second region of interest 6.

Specifically, whether the first luminescence profile and the second luminescence profile coincide with each other can be determined based on the difference between the first luminescence profile and the second luminescence profile.

According to one example, the difference between the first luminescence profile and the second luminescence profile is difference in first luminescence intensity and difference in second luminescence intensity.

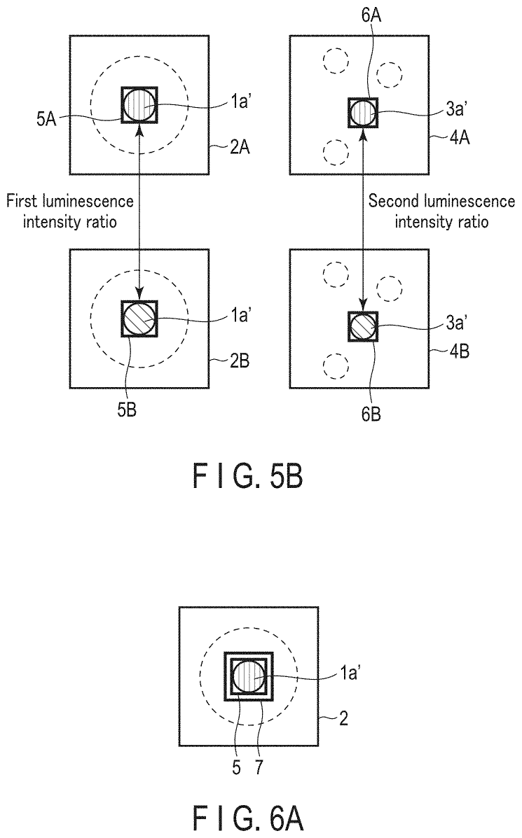

FIG. 5A is a schematic diagram showing an example of difference in first luminescence intensity, and an example of difference in second luminescence intensity. FIG. 5A illustrates the first luminescence image (color A) 2A shown in FIG. 3A, the first luminescence image (color B) 2B shown in FIG. 3B, the second luminescence image (color A) 4A shown in FIG. 4A, and the second luminescence image (color B) 4B shown in FIG. 4B.

The first luminescence profile is, for example, a luminescence intensity caused by expression of the first luminescent reporter protein (hereinafter, first luminescence intensity), and a luminescence intensity caused by expression of the second luminescent reporter protein (hereinafter, second luminescence intensity). Similarly, also the second luminescence profile is, for example, a luminescence intensity caused by expression of the first luminescent reporter protein (hereinafter, first luminescence intensity), and a luminescence intensity caused by expression of the second luminescent reporter protein (hereinafter, second luminescence intensity). As one example, a difference between the first luminescence intensity that is the first luminescence profile and the first luminescence intensity that is the second luminescence profile is divided by the first luminescence intensity that is the first luminescence profile, and the resultant value is multiplied by 100. The absolute value of the value thus obtained is defined as “difference in first luminescence intensity”. As one example, a difference between the second luminescence intensity that is the first luminescence profile and the second luminescence intensity that is the second luminescence profile is divided by the second luminescence intensity that is the first luminescence profile, and the resultant value is multiplied by 100. The absolute value of the value thus obtained is defined as “difference in second luminescence intensity”. When both the difference in first luminescence intensity and the difference in second luminescence intensity are 10% or less, it is determined that the first luminescence profile and the second luminescence profile coincide with each other.

On the other hand, when at least one of the difference in first luminescence intensity and the difference in second luminescence intensity is larger than 10%, it is determined that the first luminescence profile and the second luminescence profile do not coincide with each other. In this case, since the objective cells are specified based on the expression amount of a plurality of reprogramming factors, it is possible to specify the objective cells with higher reliability compared with the case where the objective cells are specified based on the expression amount of one reprogramming factor.

According to another example, the difference between the first luminescence profile and the second luminescence profile is, for example, difference between the first luminescence intensity ratio and the second luminescence intensity ratio.

FIG. 5B is a schematic diagram showing an example of a first luminescence intensity ratio, and an example of a second luminescence intensity ratio. FIG. 5B also illustrates the first luminescence image (color A) 2A shown in FIG. 3A, the first luminescence image (color B) 2B shown in FIG. 3B, the second luminescence image (color A) 4A shown in FIG. 4A, and the second luminescence image (color B) 4B shown in FIG. 4B.

The first luminescence intensity ratio is a luminescence intensity ratio determined from each luminescence intensity caused by expression of each of the luminescent reporter proteins, based on the first luminescence image 2. The second luminescence intensity ratio is a luminescence intensity ratio determined from each luminescence intensity caused by expression of each of the luminescent reporter proteins, based on the second luminescence image 4. In the case where the difference between the first luminescence profile and the second luminescence profile is the difference between the first luminescence intensity ratio and the second luminescence intensity ratio, it is determined that the first luminescence profile and the second luminescence profile coincide with each other when the difference between the first luminescence profile and the second luminescence profile is 10% or less. On the other hand, when the difference between the first luminescence profile and the second luminescence profile is larger than 10%, it is determined that the first luminescence profile and the second luminescence profile do not coincide with each other. In this case, since the objective cells are specified based on the ratio of expression amount of a plurality of reprogramming factors, it is possible to specify the objective cells with higher reliability compared with the case where the objective cells are specified based on the expression amount of one reprogramming factor.

In the above, another example of specifying objective cells in the plurality of second cell populations 3 based on the first luminescence image 2 and the second luminescence image 4 has been described.

According to the “[First example of specifying objective cells]”, and “[Second example of specifying objective cells]”, it is possible to specify objective cells with high reliability.

It is preferred that the first region of interest and the second region of interest have the same shape. When the shape of the first region of interest and the shape of the second region of interest are the same with each other, an error is less likely to occur in comparison between the first luminescence profile and the second luminescence profile. The fact that two regions have the same shape concretely means that two regions have the same area and the same form.

In the manner as described above, the objective cells in the plurality of second cell populations are specified.

After specifying the objective cells in the second cell population 3, the objective cells in the second cell population 3 may be cultured. Culture can be conducted in the same method as described in “<1-1. Preparation of first cell population>”.

<1-7. Other Example of Cell Specifying Method>

According to other example of the cell specifying method according to one embodiment, the selecting objective cells from the first cell population 1 based on the first luminescence image 2 includes:

selecting the first region of interest 5 concerning objective cells on the first luminescence image 2; and

selecting a third region of interest 7 concerning objective cells having an area different from an area of the first region of interest 5 on the first luminescence image 2, and

the specifying objective cells in the second cell population 3 includes:

selecting the second region of interest 6 concerning the second cell population 3 on the second luminescence image 4;

selecting a fourth region of interest 8 having an area different from an area of the second region of interest 6 concerning the second cell population 3 on the second luminescence image 4; and

specifying objective cells in the plurality of second cell populations 3 based on the first luminescence profile acquired for the first region of interest 5, the second luminescence profile acquired for the second region of interest 6, the third luminescence profile acquired for the third region of interest 7, and the fourth luminescence profile acquired for the fourth region of interest 8.

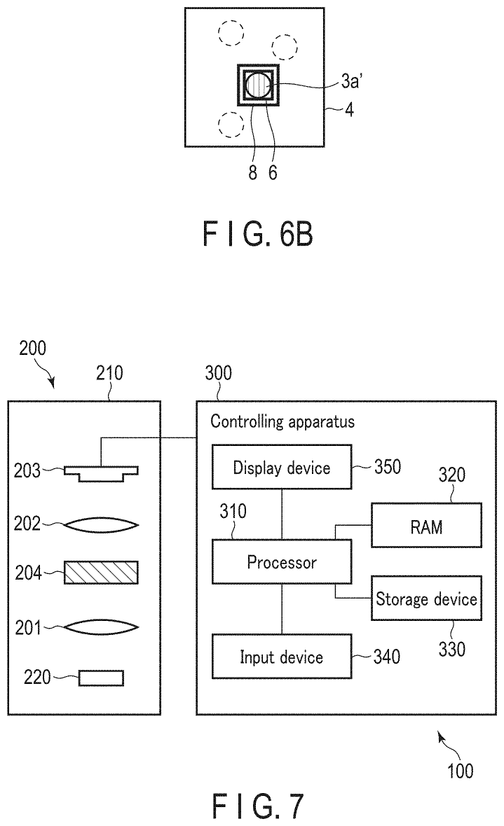

FIG. 6A is a schematic diagram showing one example of a first luminescence image in which a first region of interest and a third region of interest are selected. FIG. 6A is the first luminescence image 2 shown in S5 of FIG. 2. The third region of interest 7 is, for example, a region including the entire first region of interest 5. Alternatively, the third region of interest 7 may be, for example, a region the entirety of which is included in the first region of interest 5. The area of the third region of interest 7 is, for example, 1.1 to 100 times the area of the first region of interest 5.

FIG. 6B is a schematic diagram showing one example of a second luminescence image in which a second region of interest and a fourth region of interest are selected. FIG. 6B is the second luminescence image 4 shown in S6 of FIG. 2. The fourth region of interest 8 is, for example, a region including the entire second region of interest 6. Alternatively, the fourth region of interest 8 may be, for example, a region the entirety of which is included in the second region of interest 6. The area of the fourth region of interest 8 is, for example, 1.1 to 100 times the area of the second region of interest 6.

By evaluating the third region of interest 7 and fourth region of interest 8 based on the third luminescence profile and the fourth luminescence profile, in addition to evaluating the first region of interest 5 and the second region of interest 6 based on the first luminescence profile and the second luminescence profile, and specifying objective cells, it is possible to specify the objective cells with higher reliability compared with the case of specifying the objective cells based on only the first luminescence profile and the second luminescence profile.

Preferably, the first region of interest and the second region of interest have the same shape, and the third region of interest and the fourth region of interest have the same shape. In this case, it is possible to specify the objective cells with higher reliability.

In the above, other example of the cell specifying method has been described.

As described above, according to the cell specifying method according to one embodiment, when the first cell population containing objective cells, for example, iPS cells are divided into a plurality of second cell populations, which cells in the plurality of second cell populations are the objective cells can be easily specified.

### 2. Production Method of Cell Population

A production method of cell population according to one embodiment includes repeating the cycle including:

executing the above-described cell specifying method; and

culturing the objective cells specified by the execution to obtain a cultured cell population.

More specifically, the production method of cell population according to one embodiment includes repeating the cycle including:

executing the above-described cell specifying method; and

culturing the objective cells specified by the execution to obtain a cultured cell population, and when there is a next cycle, the cultured cell population is used as the first cell population in the next cycle.

Hereinafter, a specific example of the production method of cell population is described.

[Specific Example of Production Method of Cell Population]

Hereinafter, a specific example of the production method of cell population is described. In this example, the total number of cycles to be executed is two.

(i) Execution of Cell Specifying Method

First, the first cycle is started.

Specifically, first, the cell specifying method described in the section of “<1. Cell specifying method>” is executed. Specifically, S1 to S6 are executed. By this execution, objective cells are specified.

(ii) Culture of Objective Cells

Next, the objective cells specified in S6 are cultured to obtain a cultured cell population.

Culture of the objective cells is, for example, the culture described in the column of the above-described “<1-1. Preparation of first cell population>” of the above-described “<1. Cell specifying method>”.

Thus, the first-time cycle ends.

(iii) Execution of Cell Specifying Method

Next, the second cycle is started. Specifically, first, taking the cultured cell population as the first cell population, S2 to S6 described in the section of “<1. Cell specifying method>” are executed. By this execution, objective cells are specified.

(iv) Culture of Objective Cells

Next, the objective cells specified in S6 are cultured to obtain a cultured cell population.

Culture of the objective cells is, for example, the culture described in the column of the above-described “<1-1. Preparation of first cell population>” of the above-described “<1. Cell specifying method>”.

Thus, the second-time cycle ends.

In the above, a specific example of the production method of cell population has been described.

Preferably, the total number of cycles to be executed falls within the range of 2 to 10, and more preferably in the range of 2 to 4. When the total number of cycles to be executed falls within the range of 2 to 10, it is possible to establish a strain of the objective cells.

Also, the selecting objective cells in the n-th cycles (n is an integer of 2 or more) may include imaging a bright-field image of the first cell population, setting the center of gravity of the first cell population that is set based on the bright-field image for the cells proliferated by the culture of the n−1-th cycle, and selecting objective cells from the region within 1 mm from the center of gravity. By setting the center of gravity for the proliferated cells, and selecting objective cells from the region within 1 mm from the center of gravity, it is possible to easily establish a strain of iPS cells when the objective cells are iPS cells.

As described above, according to the production method of cell population according to one embodiment, it is possible to produce objective cells, for example, iPS cells. Also, according to the aforementioned “production method of cell population”, it is possible to culture only iPS cells. Therefore, it is conceivable that a strain of iPS cells with high purity can be established by a smaller number of times of subculture, compared with the conventional method for establishing iPS cells.

The center of gravity of cell population defined in the present description is different from the average distance from the contour of the population, but a center site of cell density in a two-dimensional or three-dimensional image inside the cell population. Since iPS cells proliferate in various directions inside one colony as a cell population, regions having different cell densities are formed in the colony regardless of the contour of the colony. According to one embodiment, by setting the center site of the cell density in the colony as the center of gravity, the object of the subculture (namely, objective cells) that is performed until the somatic cells are reprogrammed can be set in the vicinity of the center of gravity based on the cell density of the colony for each cycle. This makes it possible to realize subculture of iPS cells with high quality. Thus, in one embodiment, it is technically important to determine the position of the center of gravity based on the cell density for each cycle during the formation process until the pluripotency as the iPS cells is acquired after the early stage term of reprogramming of the somatic cells.

### 3. Luminescence Imaging System

According to another aspect, the present embodiment is capable of providing a cell specifying system for performing the above-described “cell specifying method”. Specifically, the cell specifying system is capable of acquiring the above-described first luminescence image and second luminescence image, acquiring positional information of objective cells in the first luminescence image, and specifying positional information of objective cells in the aforementioned second cell population.

FIG. 7 schematically shows one example of the above-described cell specifying system. Hereinafter, a cell specifying system is described with reference to FIG. 7. As shown in FIG. 7, a cell specifying system 100 includes a luminescence image generating apparatus 200 and a controlling apparatus 300.

The luminescence image generating apparatus 200 is an apparatus capable of acquiring an image, and quantifying the intensity of the luminescence of the sample, for example, a microscope equipped with an image pickup device or a photomultiplier. The microscope can include a luminescence imaging system LV200 (OLYMPUS Corporation).

The luminescence image generating apparatus 200 includes a black box 210. The luminescence image generating apparatus 200 detects luminescence of a sample 220 placed in the black box 210.

The luminescence image generating apparatus 200 includes, for example, an object lens 201, a tube lens 202, a detector 203, and a filter 204. The detector 203 detects light radiated from the sample 220 via the object lens 201 and the tube lens 202. The detector 203 may be an image pickup device or the like including a CCD sensor, and may be, for example, a cooled CCD, or may be a photomultiplier tube. The filter 204, for example, transmits only the light in a specific wavelength range, among the light radiated from the sample 220. The filter 204 may be, for example, an excitation light filter that separates excitation light from the light radiated from the light source. When the sample 220 contains a fluorescent protein, it is possible to excite the fluorescent protein contained in the sample 220 by the light source and the excitation light filter to emit fluorescence from the sample 220.

The filter 204 may be omitted.

The luminescence image generating apparatus 200 generates a first luminescence image and a second luminescence image. The first luminescence image is a luminescence image obtained by imaging luminescence caused by expression of a luminescent reporter protein for at least part of a first cell population containing cells into which “nucleic acid encoding reprogramming factors” and “nucleic acid encoding a luminescent reporter protein” are introduced, and containing objective cells. The first luminescence image is, for example, the above-described “first luminescence image”. The objective cells are, for example, the above-described “objective cells”. The second luminescence image is a luminescence image obtained by imaging luminescence caused by expression of a luminescent reporter protein for at least one of a plurality of second cell populations provided by division of the first cell population. The second luminescence image is, for example, the above-described “second luminescence image”.

The sample 220 is, for example, a first cell population or one or more second cell population(s).

The controlling apparatus 300 is, for example, an apparatus such as a personal computer (PC) that controls operations of the luminescence image generating apparatus 200, and analyses the data obtained in the luminescence image generating apparatus 200. The controlling apparatus 300 acquires data from the detector 203. The data obtained in the luminescence image generating apparatus 200 is, for example, a first luminescence image or a second luminescence image.

The controlling apparatus 300 includes, for example, a processor 310, a random access memory (RAM) 320, a storage device 330, an input device 340, and a display device 350.

The RAM 320 operates as a main storage device of the processor 310. The storage device 330 is, for example, a semiconductor memory, a hard disc and the like. The storage device 330 can store, a program, a parameter and the like used in the processor 310. The storage device 330 can store data obtained by using the controlling apparatus 300, an analytical result based on the data, and the like. The input device 340 can include, for example, a keyboard, a mouse, and a touch panel. The display device 350 can include, for example, a liquid crystal display.

The processor 310 performs various operations related with operations of the cell specifying system 100. The processor 310 can include, for example, a Central Processing Unit (CPU), an Application Specific Integrated Circuit (ASIC), a Field Programmable Gate Array (FPGA), or a Graphics Processing Unit (GPU). The processor 310 may be configured by one integrated circuit or the like, or may be configured by combination of a plurality of integrated circuits and the like.

The processor 310 acquires a first luminescence image and a second luminescence image based on a detection result by the detector 203. The processor 310 displays the first luminescence image and the second luminescence image on the display device 350. As the user inputs positional information of objective cells to the input device 340 based on the displayed first luminescence image, the processor 310 acquires the positional information of objective cells in the first luminescence image. The positional information of objective cells is, for example, a region concerning objective cells in the first luminescence image. The region concerning objective cells may be the above-described “first region of interest”. Hereinafter, it is assumed that the information of objective cells is the first region of interest.

The processor 310 specifies positional information of objective cells in the second luminescence image, for example, based on the first luminescence image and the second luminescence image. Specifically, for example, the following method is recited. First, the processor 310 acquires a first luminescence profile for the first region of interest. The first luminescence profile may be the above-described “first luminescence profile”. The processor 310 acquires the first luminescence profile, for example, by using existing image processing software. The processor 310 may display the information of the first region of interest and the first luminescence profile on the display device 350. Then, the processor 310 selects one or more second region (s) of interest on the second luminescence image. The second region of interest may be the above-described “second region of interest”. Then, the processor 310 acquires a second luminescence profile for the second region of interest. The second luminescence profile may be the above-described “second luminescence profile”. The processor acquires the second luminescence profile, for example, by using existing image processing software. The processor 310 may display the information of the second region of interest and the second luminescence profile on the display device 350. Then, positional information of objective cells in the second luminescence image is specified based on the first luminescence profile and the second luminescence profile. The specifying positional information of objective cells in the second luminescence image based on the first luminescence profile and one or more second luminescence profile(s) can be conducted by the same method as the method described in the section of “<1-6. Specification of objective cells>”.

As described above, according to the cell specifying system according to one embodiment, it is possible to specify which cells are objective cells in the plurality of second cell populations obtained by dividing the first cell population containing objective cells.

### 4. Other Embodiments

Other embodiments are additionally noted below.

[1]

A cell specifying method including:

for at least part of a first cell population including cells into which a nucleic acid encoding plural kinds of reprogramming factors necessary for reprogramming of somatic cells, and a nucleic acid encoding a luminescent reporter protein configured to be co-expressed with at least one kind of the plural kinds of reprogramming factors are introduced, acquiring a first luminescence image concerning luminescence caused by expression of the luminescent reporter protein;

dividing the first cell population into a plurality of second cell populations;

for at least one of the second cell populations, acquiring a second luminescence image concerning luminescence caused by expression of the luminescent reporter protein; and

specifying cells in the first cell population from cells in the plurality of second cell populations based on the first luminescence image and the second luminescence image.

[2]

The method according to [1], wherein the specifying cells in the first cell population from cells in the second cell populations based on the first luminescence image and the second luminescence image includes:

selecting a first region of interest on the first luminescence image;

acquiring a first luminescence profile for the first region of interest;

selecting a second region of interest concerning the second cell population on the second luminescence image;

acquiring a second luminescence profile for the second region of interest; and

specifying cells in the first cell population from cells in the plurality of second cell populations based on the first luminescence profile and the second luminescence profile.

### Examples

Using the techniques of the embodiments, objective cells in the second cell population obtained by dividing prepared iPS cell-like colonies were specified. The present example aims at establishing a strain of iPS cells.

<1. Preparation of First Cell Population>

Human peripheral blood mononuclear cells (PBMC; Cellular Technology Limited) were thawed, and cultured in PBMC medium (AK02 medium (AJINOMOTO CO., INC.) to which IL3, IL6, SCF, TPO, Flt-3L, and CSF are added). The cell density was 2.5×10⁶ cells/well, and the culture was conducted in a 24-well plate. After culturing the cells at 37° C., 5% CO₂ for 7 days without replacing the medium, the cells were seeded in a 6-well plate in which PBMC is coated with a coating agent (iMatrix (Nippi)). Using Amaxa (Lonza), vector A and vector B as will be described later and purchased pCE-mp53DD (Addgene), pCE-hSK (Addgene), and pCXB-EBNA1 (Addgene) were introduced into PBMC seeded in the 6-well plate. After introducing vectors into PBMC, the PBMC were cultured.

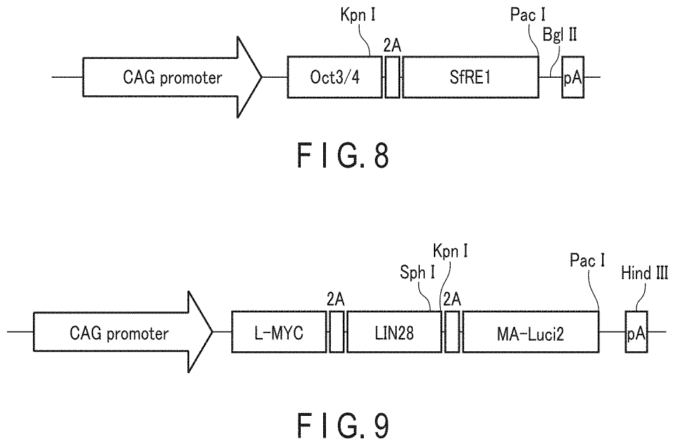

As shown in FIG. 8, a vector that is to express SfRE1 luciferase as a luminescent reporter protein together with Oct3/4 was prepared as vector A. Specifically, by incorporating SfRE1 luciferase (mam) (SEQ ID NO: 1) downstream hOCT3/4 of pCXLE-hOCT3/4-shp53 (Addgene) via a 2A sequence, vector A was prepared.

Hereinafter, a base sequence of SfRE1 luciferase is described.

(SEQ ID NO: 1) SfRE1 luciferase (mam)

atggccagcagcatgatgagcaagaaggacctggaagataagaacgtggt
gcacggccccgacccctactacctggtggatgagggcaatgccggccagc
agctgcacaagaccatcctgagatacgcccagctgcccgacacaatcgcc
ttcaccgacggccacaccaagcgggatgtgacctacgcccactacttcga
cctgacctgcagactggccgagagcctgaagagatacggcctgaacctgc
agagccggatcgccgtgtgcagcgagaacaacgtggaatttttcatcccc
gtggtggccagcctgtacctgggagtgggagtggcccccaccaacgacat
ctacaacgagacagagctgttcaacagcctgaacatcagccagcccacca
tcgtgttcgtgtccaagcgggccctgcacaagatcctggaagtgaagaag
cgcatccccatcatcaagaccgtggtggtgctggacaccgaagaggactt
catgggctaccactgcctgcacagctttatgaagcactacctgcccccca
acttcgacatcatgagctacaagcccgaagagttcgcccgggatggacag
ctggccctgatcatgaacagcagcggcagcaccggcctgcctaaaggcgt
gatgctggcccacagatccgtggtcgtgcggttcagccactgcaaggacc
ccgtgttcggcaaccagatcatccccgacaccgctatcctgaccgtgatc
cctttccaccacggcttcggcatgttcaccaccctgggctacctgacctg
tggcttccggatcgtgctgctgcggaagttcgacgagcactactttctga
agtgcctgcaggactacaagatccagtttgccctgctggtgcctaccctg
ttcagcttcttcgccaagagcaccctggtggaccagtacgacctgagcaa
cctgaaagagatcgccagcggcggagcccccctggctaaagaagtgggag
aggccgtcgccaagcggtttaagctgcccggcatcagacagggctacggc
ctgaccgagacaaccagcgccgtgatcatcacccccgagggcgaggataa
gcctggctctacaggcaaggtggtgccattcttcagcgccaagatcgtgg
acctgaacagcggcaagagcgtgggccctcaccagaggggagaactctac
ctgaagggcgacatgatcatgatgggctactgcaacaacaaggccgccac
cgacgagatgatcgacaaggatggctggctgcactccggcgacgttgcct
actacgacgaggacggccacttcttcatcgtggaccggctgaagtccctg
atcaagtacaagggctaccaggtggcccctgccgaactggaagctgtgct
gctgcagcatccctgcatcttcgatgccggcgtgaccggcgtgccagatg
atgtggacggcgaactgcctggcgcctgtgtggtcctggaaaagggcaag
cacgtgaccgagcaggaagtgatggactacgtcgccggccagctgagctg
ctacaagagactgagaggcggtgtgcgcttcatcgatgagatccctaagg
gcctgaccggcaagatcgaccggaaggccctgaaagaaatcctgaagaaa
ccccagagcaagatgtga

As shown in FIG. 9, a vector that is to express MA-Luci2 luciferase as a luminescent reporter protein together with L-myc and LIN28 was prepared as vector B. Specifically, by incorporating MA-Luci2 luciferase (mam) (SEQ ID NO: 2) downstream LIN28 of pCXLE-hUL (Addgene) via a 2A sequence, vector B was prepared. SfRE1 luciferase shows red light having a maximum luminescence wavelength λmax=610 nm, and MA-Luci2 luciferase shows green light having a maximum luminescence wavelength λmax=560 nm.

Hereinafter, a base sequence of MA-Luci2 luciferase is described.

(SEQ ID NO: 2) MA-Luci2 luciferase (mam)

atggacaagaacatcatctacggccctccccccgtgtaccccctggatgatggcaca
ggc
ggcgagcagctgtacaagtgcatcctgagatacgccaagatccccgagtgcgtggcc
ctg
accagcgcccacaccaaagagagcatcctgtacgaggaactgctgcagctgacctgc
aag
ctggcccagagcctgaagagatgcggcatcacccggaacagcacaatcgccgtgtgc
ago
gagaacaacctgcagtacttcatccccatcattgccggcctgtacatcggagccgcc
aca
gccgccgtgaacaaccggtacaacgagagagagctgaccgacatcctgaacctgagc
aag
cccgacatcatcttttgctccaaagagacactgcccaagatctgccaggtcaagaag
aag
ctgaactacatcaaagaaatcatcgtgctggacagcaagcacgacagcgagctggct
cag
tgtctggacaacttcatcagccacaactgcaacaaggacttcgacgcctaccagttc
aag
cccagcagcttcaaccggaacgaacaggtcggcctgatcctgaacagcagcggcagc
acc
ggcctgcccaagggcgtgatgctgacccacaagaacctggtggtgcgcttcagccac
tgc
aaggaccccgtgttcggcaacatcatcagccccggcaccgccatcctgaccgtgatc
cot
ttccaccacggcttcggcatgttcaccaccctgggctacttcacctgtggcttccgg
atc
gtgctgatgcacaccttctacgagaagctgttcctgcaggccctggaagattacaag
gtg
gaaagcaccctgctggtgcctaccctgatgaccttcttcgccaagagcgccctggtg
gac
aagtacaacctgccctacctgaaagagatcgccagcggcggagcccccctgagcaaa
gaa
atcggcgaggccgtggccagacggttcaagctgaacgccatccggcagggctacggc
ctg
accgagacaaccagcgccgtgctgatcacccccgagagcgagacagtgcccggcagc
atc
ggcaaggtggtgccattcttcgccgccaagatcatcgaccaccggaccggcaaggcc
ctg
ggccctaatgaagtgggcgagctgtgcttcaagggcgacatgatcatgaagggctac
tgc
aacaacatcgaggccaccaacgccatcatcgacaacgacggctggctgcacagcggc
gat
ctgggctactacaacgacgacaagcacttcttcatcgtggaccggctgaagtccatc
atc
aagtacaagggctaccaggtggcccctgccgagctggaaggcatcctgctgacacac
ccc
agcatcatggatgccggcgtgaccggcatccccgacgataatgccggcgagctgcct
gcc
gcctgcgtggtggtgaaacccggcagacacctgaccgaggaaaacgtgatcaactac
gtg
tccagccaggtgtccagcgtgaagcggctgagaggcggcgtgcggttcctggacgag
atc
cctaagggctccaccggcaagatcgacaccaccgccctgaagcagatcctgcagaag
ccc
aactgcaagctgtga

Two days or later after introduction of vectors, 1.5 ml of iPS cell medium (AK02 medium (AJINOMOTO CO., INC.)) was added every other day. At eighth day after introduction of vectors, the whole culture medium was sucked and 2 ml of iPS cell medium was added. After this, the culture medium was replaced with 2 ml of iPS cell medium every other day. After formation of iPS cell-like colonies, D-luciferin (Promega Corporation) was added to the culture medium in a final concentration of 1 mM. Here, the iPS cell-like colony is also called a first cell population.

<2. Acquisition of First Luminescence Image Using Luminescence Imaging Method>

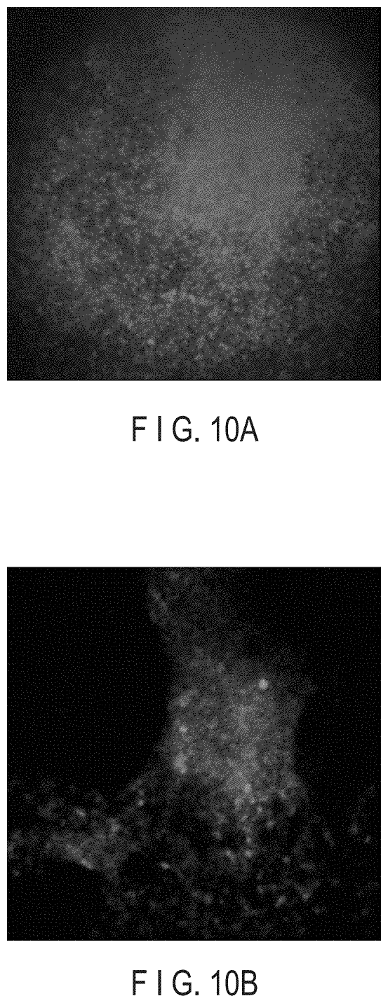

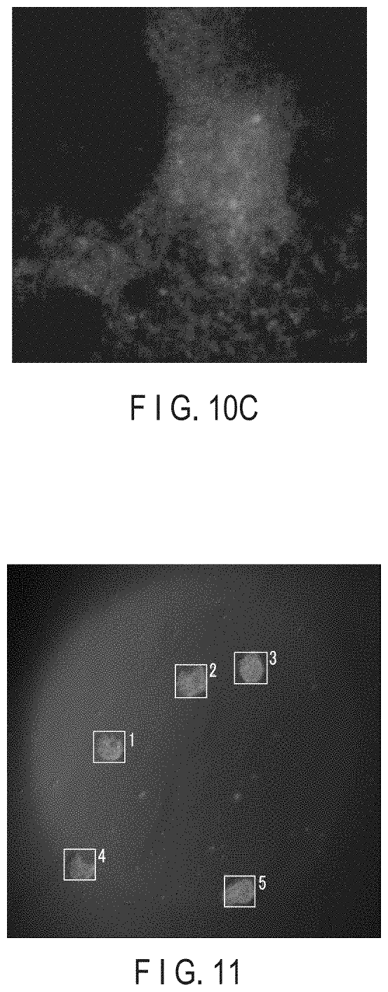

FIG. 10A is a phase contrast image showing a first cell population. FIG. 10B is a first luminescence image (green) showing luminescence originating in MA-Luci2 luciferase. FIG. 10C is a first luminescence image (red) showing luminescence originating in SfRE1 luciferase.

Imaging of the luminescence image of the iPS cell-like colony was performed using a luminescence microscope system LV200 (OLYMPUS Corporation). A phase contrast image, a first luminescence image (red) and a first luminescence image (green) were acquired using a 20× object lens LUC PlanFLN (OLYMPUS Corporation). For acquisition of image, an EM-CCD camera, ImagEM (Hamamatsu Photonics K.K.) was used, and the exposure time was 200 msec for acquisition of a phase contrast image, and 10 sec for acquisition of a luminescence image. In imaging of a luminescence image, BP495-540 (Filter1), and 610ALP (Filter2) were respectively used as a measurement filter so as to separate the green light derived from MA-Luci2 luciferase and the red light derived from SfRE1 luciferase.

<3. Division of First Cell Population>

FIG. 11 is a phase contrast image showing a second cell population.

The colony center part was selected from the acquired phase contrast observation image, and the colony center part was picked up with a PIPETMAN P-10 (Gilson Incorporated) under observation with a stereo microscope. The colony center part included the later-described first region of interests (No. 1) to (No. 15). After picking up of the colony, the colony was divided into small masses of cells each consisting of several to several tens cells by pipetting with care not to divide into a single cell state, and discharged to a 6-well plate. The small mass of cells was taken as the second cell population.

<4. Acquisition of Second Luminescence Image>

A phase contrast image and a luminescence image of small masses of cells seeded in 6-well plate after division of colony were acquired. Acquisition of the second luminescence image was conducted in the same conditions as those at the time of acquiring the first luminescence image.

<5. Selection of Objective Cells>

Based on the first luminescence image, objective cells were selected.

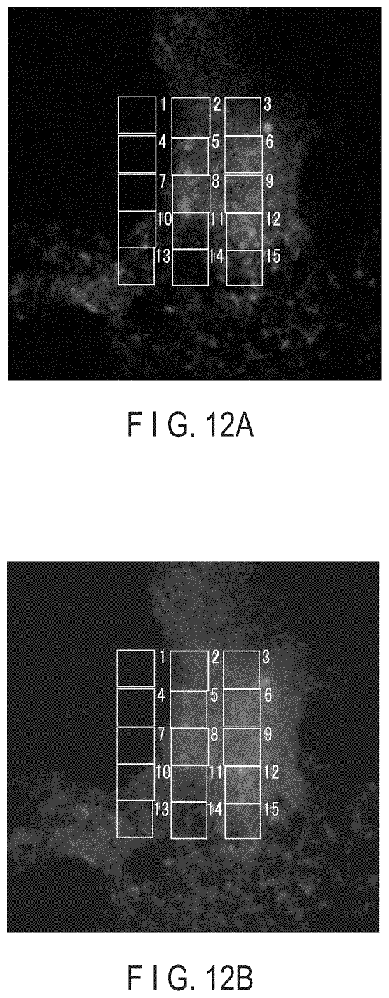

FIG. 12A is a first luminescence image (green) shown in FIG. 10B in which a first region of interest (green) is selected. As shown in FIG. 12A, fifteen first regions of interest (green) were selected in the region chipped with a PIPETMAN in the first luminescence image (green) as objective cells. The fifteen first regions of interest (green) were named first regions of interest (green) (No. 1) to (No. 15), respectively.

FIG. 12B is a first luminescence image (red) shown in FIG. 10C in which a first region of interest (red) is selected. As shown in FIG. 12B, fifteen first regions of interest (red) were selected on the first luminescence image (red). The fifteen first regions of interest (red) were named first regions of interest (red) (No. 1) to (No. 15), respectively. In the following, the region of interest is also called ROI.

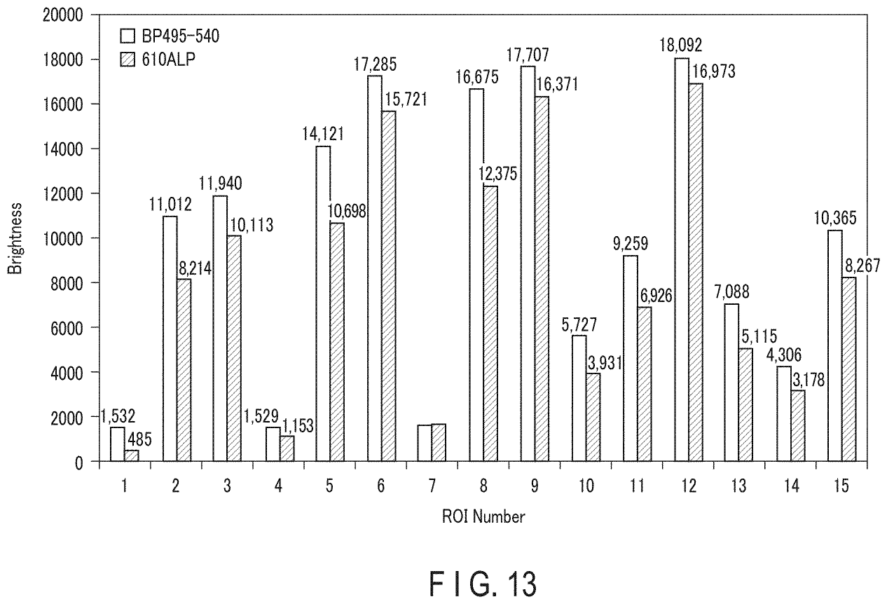

FIG. 13 is a graph showing brightness (luminescence intensity) acquired for each of the first regions of interest (red) (610ALP) and (green) (BP495-540). Such a luminescence intensity was taken as a first luminescence profile. Luminescence intensity analysis in the first regions of interest (green) (No. 1) to (No. 15) and the first regions of interest (red) (No. 1) to (No. 15) was conducted by brightness analysis for each designated region by cellSens (OLYMPUS Corporation) which is image analysis software, while following the motion of living cells.

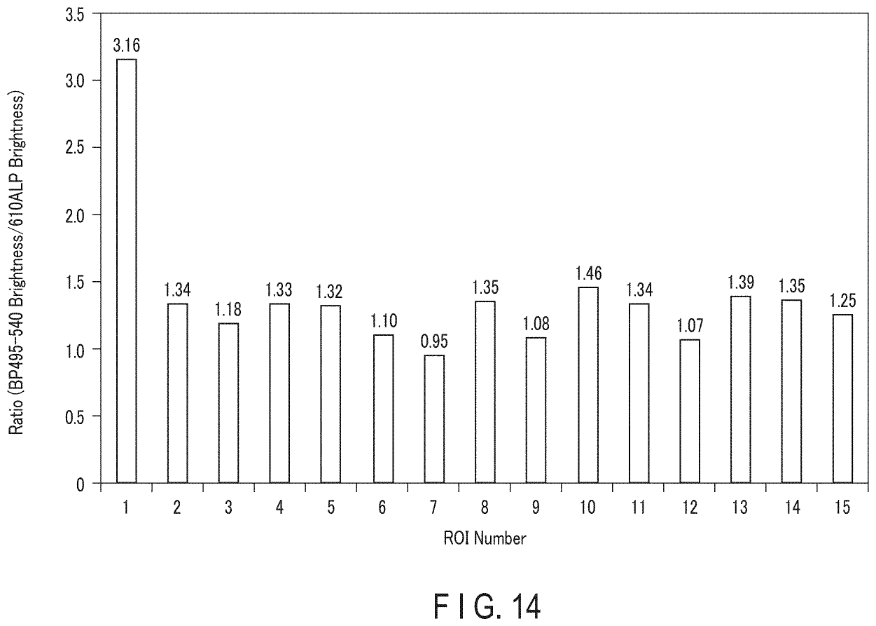

FIG. 14 is a graph showing first luminescence intensity ratio (Ratio (BP495-540 brightness/610ALP brightness)) determined from brightness (luminescence intensity) acquired for each of the first regions of interest (red) and (green). A ratio between the luminescence intensity of the green component separated by the BP495-540 filter, and the luminescence intensity of the red component separated by the 610ALP filter was calculated.

<6. Specification of Objective Cells>

Based on the first luminescence image and the second luminescence image, objective cells were specified.

First, in the second luminescence image, second regions of interest were selected.

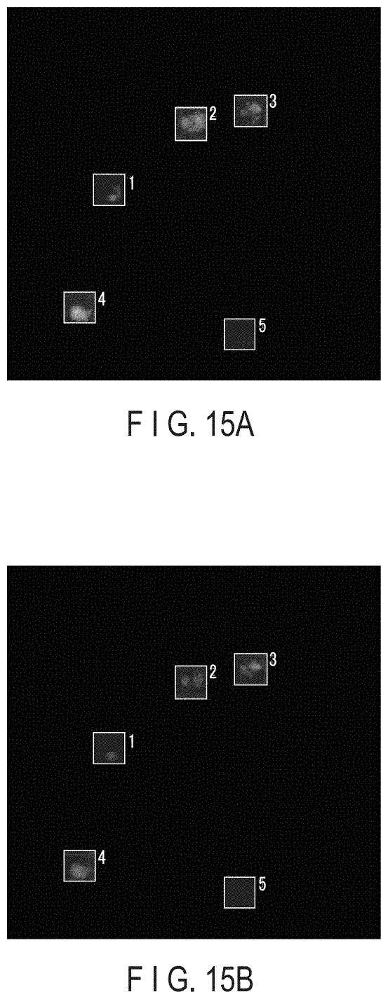

FIG. 15A is a second luminescence image (green) showing luminescence originating in MA-Luci2 luciferase in which the second region of interest (green) is selected. FIG. 15B is a second luminescence image (red) showing luminescence originating in SfRE1 luciferase in which the second region of interest (red) is selected.

At this time, the second region of interest was selected so that the size and the shape of the second region of interest is equivalent to those of the first region of interest. Second regions of interest were selected for each of five small masses of cells observed in the second luminescence image.

Specifically, five second regions of interest (green) were selected in the second luminescence image (green). The five second regions of interest (green) were named second regions of interest (green) (No. 1) to (No. 5), respectively. Then, five second regions of interest (red) were selected on the second luminescence image (red). The five second regions of interest (red) were named second regions of interest (red) (No. 1) to (No. 5), respectively.

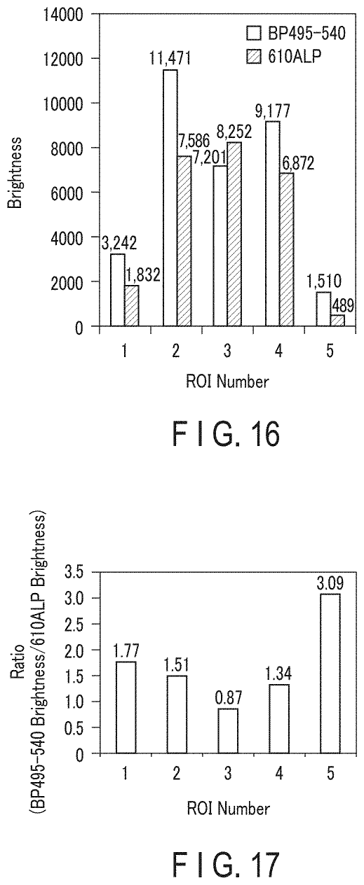

FIG. 16 is a graph showing brightness (luminescence intensity) acquired for each of the second regions of interest (red) (610ALP) and (green) (BP495-540). Such a luminescence intensity was taken as a second luminescence profile. Luminescence intensity analysis in each of the second regions of interest (green) (No. 1) to (No. 5) and the second regions of interest (red) (No. 1) to (No. 5) was conducted using cellSens (OLYMPUS Corporation).

FIG. 17 is a graph showing second luminescence intensity ratio (Ratio (BP495-540 brightness/610ALP brightness)) determined from brightness (luminescence intensity) acquired for each of the second regions of interest (red) and (green). The second luminescence intensity ratio was calculated based on the luminescence intensity of the green component separated by the BP495-540 filter, and the luminescence intensity of the red component separated by the 610ALP filter.

Next, based on the first luminescence profile acquired for the first region of interest, and the second luminescence profile acquired for the second region of interest, objective cells in the second cell population were specified.

Table 3 indicates difference between each first region of interest (green) and each second region of interest (green). In Table 3, “*” indicates that the difference between the luminescence intensity of the first region of interest (green) and the luminescence intensity of the second region of interest (green) is within 10%.

TABLE 3

| | | First region of interest (green) | | | | | | | |
|---|---|---|---|---|---|---|---|---|---|
| | | (1) | (2) | (3) | (4) | (5) | (6) | (7) | (8) |
| Second region of | (1) | 111.61 | 70.56 | 72.85 | 112.00 | 77.04 | 81.24 | 100.84 | 80.56 |
| interest (green) | (2) | 648.74 | *4.16 | *3.93 | 650.11 | 18.77 | 33.64 | 610.61 | 31.21 |
| | (3) | 370.04 | 34.61 | 39.69 | 370.90 | 49.01 | 58.34 | 346.10 | 56.82 |
| | (4) | 499.00 | 16.67 | 23.14 | 500.10 | 35.01 | 46.91 | 468.50 | 44.97 |
| | (5) | *1.41 | 86.28 | 87.35 | *1.23 | 89.30 | 91.26 | *6.43 | 90.94 |

| | | First region of interest (green) | | | | | | |
|---|---|---|---|---|---|---|---|---|
| | | (9) | (10) | (11) | (12) | (13) | (14) | (15) |
| Second region of | (1) | 81.69 | 43.39 | 64.99 | 82.08 | 54.26 | 24.71 | 68.72 |
| interest (green) | (2) | 35.22 | 100.29 | 23.88 | 36.60 | 61.83 | 166.38 | 10.67 |
| | (3) | 59.33 | 25.73 | 22.23 | 60.20 | *1.59 | 67.22 | 30.53 |
| | (4) | 48.18 | 60.23 | *0.89 | 49.28 | 29.47 | 113.11 | 11.47 |
| | (5) | 91.47 | 73.63 | 83.69 | 91.65 | 78.69 | 64.93 | 85.43 |

Table 4 indicates difference between each first region of interest (red) and each second region of interest (red). In Table 4, “*” indicates that the difference between the luminescence intensity of the first region of interest (red) and the luminescence intensity of the second region of interest (red) is within 10%.

TABLE 4

| | | First region of interest (red) | | | | | | | |
|---|---|---|---|---|---|---|---|---|---|
| | | (1) | (2) | (3) | (4) | (5) | (6) | (7) | (8) |
| Second region of | (1) | 277.78 | 77.69 | 81.88 | 58.88 | 82.87 | 88.35 | *7.58 | 85.19 |
| interest (red) | (2) | 1464.07 | *7.65 | 24.99 | 557.80 | 29.09 | 51.75 | 345.38 | 38.70 |
| | (3) | 1601.39 | *0.46 | 18.41 | 615.55 | 22.87 | 47.51 | 384.48 | 33.32 |
| | (4) | 1316.83 | 16.34 | 32.05 | 495.88 | 35.77 | 56.29 | 303.45 | 44.47 |
| | (5) | *0.85 | 94.05 | 95.16 | 57.58 | 95.43 | 96.89 | 71.28 | 96.05 |

| | | First region of interest (red) | | | | | | |
|---|---|---|---|---|---|---|---|---|
| | | (9) | (10) | (11) | (12) | (13) | (14) | (15) |
| Second region of | (1) | 88.81 | 53.39 | 73.55 | 89.21 | 64.18 | 42.35 | 77.84 |
| interest (red) | (2) | 53.66 | 92.96 | *9.52 | 55.31 | 48.30 | 138.68 | *8.24 |
| | (3) | 49.60 | 109.90 | 19.14 | 51.38 | 61.32 | 159.64 | *0.19 |
| | (4) | 58.03 | 74.80 | *0.79 | 59.51 | 34.34 | 116.21 | 16.88 |
| | (5) | 97.01 | 87.56 | 92.94 | 97.12 | 90.44 | 84.61 | 94.08 |

Table 5 shows correspondence between the first region of interest and the second region of interest.

TABLE 5

| First region of | Second region of |
|---|---|
| interest | interest |
| (1) | (5) |
| (2) | (2) |
| (3) | No correspondence |
| (4) | No correspondence |
| (5) | No correspondence |
| (6) | No correspondence |
| (7) | No correspondence |
| (8) | No correspondence |
| (9) | No correspondence |
| (10) | No correspondence |
| (11) | (4) |
| (12) | No correspondence |
| (13) | No correspondence |
| (14) | No correspondence |
| (15) | No correspondence |

Here, focusing on both of the green component and the red component, the luminescence intensity of the first region of interest and the luminescence intensity of the second region of interest were compared. In comparison, the difference between the luminescence intensity of each first region of interest and the luminescence intensity of each second region of interest was divided by the luminescence intensity of the first region of interest, and the resultant value was multiplied by 100, and the absolute value of the resultant value was calculated. The regions of interest for which the difference is calculated to be within 10% in both of the green component and the red component can be determined to have correspondence. Also, from the result of analysis of luminescence intensity ratio using FIG. 14 and FIG. 17, the same result as in Table 5 can be obtained.

As shown in Table 5, it was revealed that the first region of interest (No. 1) and second region of interest (No. 5) correspond with each other, the first region of interest (No. 2) and the second region of interest (No. 2) correspond with each other, and the first region of interest (No. 11) and the second region of interest (No. 4) correspond with each other.

<7. Culture of Objective Cells>

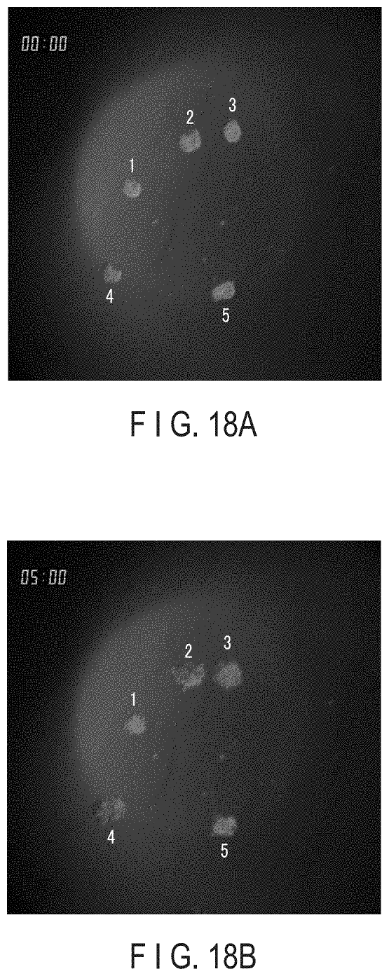

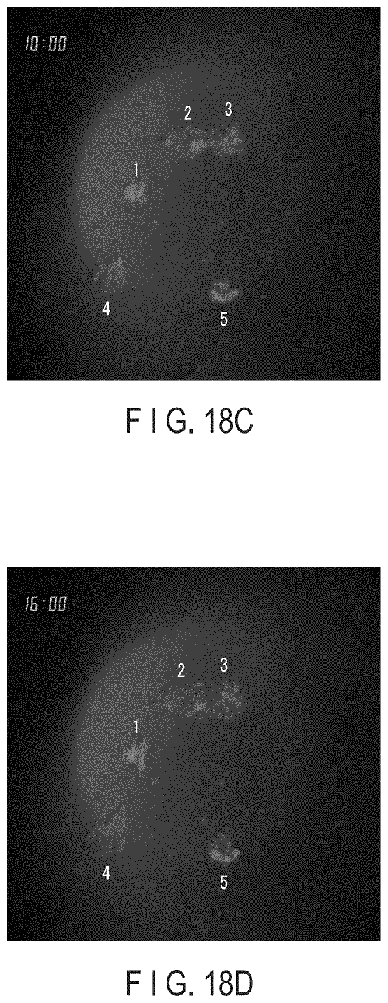

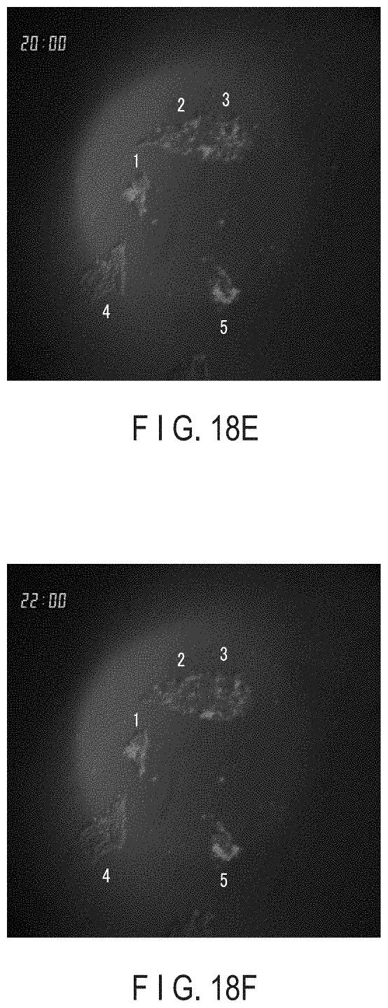

FIG. 18A is a bright-field image showing objective cells at the time of starting the culture. FIG. 18B to FIG. 18F each show a bright-field image showing objective cells after a lapse of a predetermined time from culture of the objective cells. FIG. 18B to FIG. 18F were imaged after 5, 10, 16, 20, and 22 hours from the point of time at which culture started.

After specifying objective cells in the plurality of second cell populations, the objective cells were cultured. In association with contiguous culture, even the small mass of cells (2) contained in the second region of interest (No. 2) for which correspondence has been obtained sometimes bind with the small mass of cells (3) contained in the second region of interest (No. 3) and can no longer be separated. In this case, it is possible to establish a strain of iPS cells with high purity by culturing the small mass of cells (4) contained in the second region of interest (No. 4) or the small mass of cells (5) contained in the second region of interest (No. 5) for which correspondence has been obtained until the mass reaches a sufficient size and picking up the mass.

As shown in the above, by using embodiments, it is possible to easily specify which cells are objective cells in the plurality of second cell populations obtained by dividing the first cell population. By culturing only objective cells, it becomes possible to easily obtain a strain of iPS cells with high purity.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A cell specifying method comprising:
for at least part of a first cell population including cells into which a nucleic acid encoding plural kinds of reprogramming factors necessary for reprogramming of somatic cells, and a nucleic acid encoding a luminescent reporter protein configured to be co-expressed with at least one kind of the plural kinds of reprogramming factors are introduced, acquiring a first luminescence image concerning luminescence caused by expression of the luminescent reporter protein;
dividing the first cell population into a plurality of second cell populations;
for at least one of the second cell populations, acquiring a second luminescence image concerning luminescence caused by expression of the luminescent reporter protein;
selecting objective cells from the first cell population based on the first luminescence image; and
specifying the objective cells in the plurality of second cell populations based on the first luminescence image and the second luminescence image.

2. The method according to claim 1, wherein the luminescent reporter protein is a bioluminescent reporter protein or a fluorescent reporter protein.

3. The method according to claim 1, wherein
the selecting objective cells includes selecting a first region of interest concerning the objective cells on the first luminescence image, and
the specifying the objective cells in the plurality of second cell populations includes:
selecting a second region of interest concerning the second cell population on the second luminescence image; and
specifying the objective cells in the plurality of second cell populations based on a first luminescence profile acquired for the first region of interest and a second luminescence profile acquired for the second region of interest.

4. The method according to claim 1, wherein
the selecting the objective cells includes: selecting a first region of interest concerning the objective cells on the first luminescence image; and
selecting a third region of interest concerning the objective cells on the first luminescence image, the third region of interest having an area different from an area of the first region of interest, and
the specifying the objective cells in the plurality of second cell populations includes:
selecting a second region of interest concerning the second cell population on the second luminescence image;
selecting a fourth region of interest on the second luminescence image, the fourth region of interest having an area different from the area of the second region of interest concerning the second cell population; and
specifying the objective cells in the plurality of second cell populations based on a first luminescence profile acquired for the first region of interest, a second luminescence profile acquired for the second region of interest, a third luminescence profile acquired for the third region of interest, and a fourth luminescence profile acquired for the fourth region of interest.

5. The method according to claim 3, wherein the first region of interest and the second region of interest have the same shape.

6. The method according to claim 4, wherein the first region of interest and the second region of interest have the same shape, and the third region of interest and the fourth region of interest have the same shape.

7. The method according to claim 3, wherein the first region of interest includes three to ten of the cells.

8. The method according to claim 3, wherein
among the plural kinds of reprogramming factors, the reprogramming factors that are co-expressed with the luminescent reporter protein are two or more kinds,
each of the luminescent reporter proteins co-expressed with the two or more kinds of reprogramming factors has such luminescence characteristics that allow detection of a protein distinguishably from any other luminescent reporter proteins,
the first luminescence profile is luminescence intensity caused by expression of each of the luminescent reporter proteins, and
the second luminescence profile is luminescence intensity caused by expression of each of the luminescent reporter protein.

9. The method according to claim 1, wherein
among the plural kinds of reprogramming factors, the reprogramming factors co-expressed with the luminescent reporter protein are two or more kinds, and
each of the luminescent reporter proteins co-expressed with the two or more kinds of reprogramming factors has such luminescence characteristics that allow detection of a protein distinguishably from any other luminescent reporter proteins.

10. The method according to claim 1, wherein the first luminescence image is one image that is selected from a plurality of luminescence images, and
each of the plurality of luminescence images is an image showing a plane at a position that is different from a position of any other luminescence images along a thickness direction of the first cell population.

11. The method according to claim 1, wherein the selecting objective cells from the first cell population includes selecting the objective cells from a center site of cell density as a center of gravity of the first cell population.

12. The method according to claim 1, wherein the selecting the objective cells from the first cell population includes imaging a bright-field image of the first cell population, and selecting the objective cells from a region within 1 mm from a center of gravity of the first cell population that is set based on the bright-field image.

13. The method according to claim 1, wherein the reprogramming factor co-expressed with the luminescent reporter protein is at least one selected from the group consisting of Oct3/4, Klf4, Sox2, c-myc, Lin28, and L-myc.

14. The method according to claim 1, wherein the nucleic acid encoding the reprogramming factor and the nucleic acid encoding the luminescent reporter protein are introduced into the cells in a form of an episomal vector.

15. The method according to claim 1, wherein the second cell population includes three to ten cells.

16. A production method of cell population, comprising repeating a cycle, the cycle including:
executing the method according to claim 1; and
culturing the objective cells specified by the execution to obtain a cultured cell population.

17. The method according to claim 16, wherein the selecting the objective cells from the first cell population includes selecting the objective cells from a center site of cell density as a center of gravity of the first cell population.

18. The method according to claim 16, wherein the selecting objective cells in the n-th cycle (n is an integer of 2 or more) includes imaging a bright-field image of the first cell population, setting a center of gravity of the first cell population that is set based on the bright-field image for a cell proliferated by the culture of the n−1-th cycle, and selecting objective cells from a region within 1 mm from the center of gravity.

19. A cell specifying system comprising:
a luminescence image generating apparatus configured to generate a first luminescence image concerning luminescence caused by expression of a luminescent reporter protein for at least part of a first cell population including cells into which a nucleic acid encoding plural kinds of reprogramming factors necessary for reprogramming of somatic cells and a nucleic acid encoding the luminescent reporter protein configured to be co-expressed with at least one kind of the plural kinds of reprogramming factors are introduced, and a second luminescence image concerning luminescence caused by expression of the luminescent reporter protein for at least one of a plurality of second cell populations obtained by dividing the first cell population; and
a processor configured to perform acquiring the first luminescence image and the second luminescence image from the luminescence image generating apparatus, selecting objective cells from the first cell population based on the first luminescence image, acquiring positional information of the objective cells in the first luminescence image, and specifying positional information of the objective cells in the second luminescence image based on the first luminescence image and the second luminescence image.

20. The cell specifying system according to claim 19, wherein
the acquiring positional information of the objective cells includes:
acquiring a first region of interest selected on the first luminescence image as positional information of the objective cells, and
the specifying positional information of the objective cells includes:
selecting a second region of interest concerning the second cell population on the second luminescence image; and
specifying positional information of the objective cells in the second luminescence image based on the first luminescence profile acquired for the first region of interest and the second luminescence profile acquired for the second region of interest.


SEQUENCE LISTING (2 sequences)

SEQ ID NO: 1 - DNA, 1668 bp, Artificial Sequence
  SfRE1 luciferase (mam)
atggccagcagcatgatgagcaagaaggacctggaagataagaacgtggtgcacggccccgacccctactacctggtggatgagggcaatgccggccagcagctgcacaagaccatcctgagatacgcccagctgcccgacacaatcgccttcaccgacggccacaccaagcgggatgtgacctacgcccactacttcgacctgacctgcagactggccgagagcctgaagagatacggcctgaacctgcagagccggatcgccgtgtgcagcgagaacaacgtggaatttttcatccccgtggtggccagcctgtacctgggagtgggagtggcccccaccaacgacatctacaacgagacagagctgttcaacagcctgaacatcagccagcccaccatcgtgttcgtgtccaagcgggccctgcacaagatcctggaagtgaagaagcgcatccccatcatcaagaccgtggtggtgctggacaccgaagaggacttcatgggctaccactgcctgcacagctttatgaagcactacctgccccccaacttcgacatcatgagctacaagcccgaagagttcgcccgggatggacagctggccctgatcatgaacagcagcggcagcaccggcctgcctaaaggcgtgatgctggcccacagatccgtggtcgtgcggttcagccactgcaaggaccccgtgttcggcaaccagatcatccccgacaccgctatcctgaccgtgatccctttccaccacggcttcggcatgttcaccaccctgggctacctgacctgtggcttccggatcgtgctgctgcggaagttcgacgagcactactttctgaagtgcctgcaggactacaagatccagtttgccctgctggtgcctaccctgttcagcttcttcgccaagagcaccctggtggaccagtacgacctgagcaacctgaaagagatcgccagcggcggagcccccctggctaaagaagtgggagaggccgtcgccaagcggtttaagctgcccggcatcagacagggctacggcctgaccgagacaaccagcgccgtgatcatcacccccgagggcgaggataagcctggctctacaggcaaggtggtgccattcttcagcgccaagatcgtggacctgaacagcggcaagagcgtgggccctcaccagaggggagaactctacctgaagggcgacatgatcatgatgggctactgcaacaacaaggccgccaccgacgagatgatcgacaaggatggctggctgcactccggcgacgttgcctactacgacgaggacggccacttcttcatcgtggaccggctgaagtccctgatcaagtacaagggctaccaggtggcccctgccgaactggaagctgtgctgctgcagcatccctgcatcttcgatgccggcgtgaccggcgtgccagatgatgtggacggcgaactgcctggcgcctgtgtggtcctggaaaagggcaagcacgtgaccgagcaggaagtgatggactacgtcgccggccagctgagctgctacaagagactgagaggcggtgtgcgcttcatcgatgagatccctaagggcctgaccggcaagatcgaccggaaggccctgaaagaaatcctgaagaaaccccagagcaagatgtga

SEQ ID NO: 2 - DNA, 1635 bp, Artificial Sequence
  MA-Luci2 luciferase (mam)
atggacaagaacatcatctacggccctccccccgtgtaccccctggatgatggcacaggcggcgagcagctgtacaagtgcatcctgagatacgccaagatccccgagtgcgtggccctgaccagcgcccacaccaaagagagcatcctgtacgaggaactgctgcagctgacctgcaagctggcccagagcctgaagagatgcggcatcacccggaacagcacaatcgccgtgtgcagcgagaacaacctgcagtacttcatccccatcattgccggcctgtacatcggagccgccacagccgccgtgaacaaccggtacaacgagagagagctgaccgacatcctgaacctgagcaagcccgacatcatcttttgctccaaagagacactgcccaagatctgccaggtcaagaagaagctgaactacatcaaagaaatcatcgtgctggacagcaagcacgacagcgagctggctcagtgtctggacaacttcatcagccacaactgcaacaaggacttcgacgcctaccagttcaagcccagcagcttcaaccggaacgaacaggtcggcctgatcctgaacagcagcggcagcaccggcctgcccaagggcgtgatgctgacccacaagaacctggtggtgcgcttcagccactgcaaggaccccgtgttcggcaacatcatcagccccggcaccgccatcctgaccgtgatccctttccaccacggcttcggcatgttcaccaccctgggctacttcacctgtggcttccggatcgtgctgatgcacaccttctacgagaagctgttcctgcaggccctggaagattacaaggtggaaagcaccctgctggtgcctaccctgatgaccttcttcgccaagagcgccctggtggacaagtacaacctgccctacctgaaagagatcgccagcggcggagcccccctgagcaaagaaatcggcgaggccgtggccagacggttcaagctgaacgccatccggcagggctacggcctgaccgagacaaccagcgccgtgctgatcacccccgagagcgagacagtgcccggcagcatcggcaaggtggtgccattcttcgccgccaagatcatcgaccaccggaccggcaaggccctgggccctaatgaagtgggcgagctgtgcttcaagggcgacatgatcatgaagggctactgcaacaacatcgaggccaccaacgccatcatcgacaacgacggctggctgcacagcggcgatctgggctactacaacgacgacaagcacttcttcatcgtggaccggctgaagtccatcatcaagtacaagggctaccaggtggcccctgccgagctggaaggcatcctgctgacacaccccagcatcatggatgccggcgtgaccggcatccccgacgataatgccggcgagctgcctgccgcctgcgtggtggtgaaacccggcagacacctgaccgaggaaaacgtgatcaactacgtgtccagccaggtgtccagcgtgaagcggctgagaggcggcgtgcggttcctggacgagatccctaagggctccaccggcaagatcgacaccaccgccctgaagcagatcctgcagaagcccaactgcaagctgtga